(19) United States
(12) Patent Application Publication
(10) Pub. No.: US 2020/0375975 A1
(43) Pub. Date: Dec. 3, 2020

(54) Treatment of Renal Cell Carcinoma with Lenvatinib and Everolimus

(71) Applicant: Eisai R&D Management Co., Ltd., Tokyo (JP)

(72) Inventors: Alton KREMER, Woodcliff Lake, NJ (US); Corina DUTCUS, Woodcliff Lake, NJ (US)

(73) Assignee: Eisai R&D Management Co., Ltd., Tokyo (JP)

(21) Appl. No.: 16/997,378

(22) Filed: Aug. 19, 2020

Related U.S. Application Data
(63) Continuation-in-part of application No. 16/092,245, filed on Oct. 9, 2018.
(60) Provisional application No. 62/322,916, filed on Apr. 15, 2016.

(30) Foreign Application Priority Data
Apr. 15, 2016 (JP) 2016-081787

(51) Int. Cl. A61K 31/47; A61K 31/436; A61P 35/00

(52) U.S. Cl. CPC A61K 31/47; A61P 35/00; A61K 31/436

(57) ABSTRACT

Methods for treating a renal cell carcinoma with an improved combination of lenvatinib or a pharmaceutically acceptable salt thereof and everolimus are provided. These methods further comprises adjusting the dosages of lenvatinib during the onset of adverse effects to lead to improved treatment methods for human subjects with renal cell carcinoma. Particularly useful dosages and dose modifications upon the occurrence of adverse events are also provided.

### CROSS-REFERENCE

This application is a continuation in part application of U.S. patent application Ser. No. 16/092,245, which is the United States national stage under 35 U.S.C. Section 371 of PCT International Patent Application No. PCT/JP2017/015461, filed on Apr. 17, 2017, which is a claims the benefit of U.S. Provisional Patent Application No. 62/322,916 and Japanese Patent Application No. JP2016-081787, both filed on Apr. 15, 2016, the entire contents of each being incorporated herein by reference.

### TECHNICAL FIELD

The present application relates generally to methods of treating renal cell carcinoma.

### BACKGROUND OF THE INVENTION

Kidney cancer constitutes approximately 3% of all cancers worldwide and is among the 10 most common cancers in both men and women. Overall, the lifetime risk for developing kidney cancer is about 1.6%, with the risk being higher in men than in women. The American Cancer Society estimates that in 2016 there will be about 62,700 new cases (39,650 in males and 23,050 in females) of kidney cancer diagnosed in the United States with about 14,240 deaths (9,240 men and 5,000 women).

Renal cell carcinoma (RCC) represents on average over 90% of all malignancies of the kidney that occur in adults. RCC arises from the epithelium of the renal tubules; specifically, it originates within the renal cortex from the proximal renal tubular epithelium. RCC has a male-to-female preponderance of 1.6:1 and is most common in those aged 40-70 years. The incidence of RCC is greater in people of Northern European ancestry and North Americans than in those of Asian or African descent.

Approximately 40% of patients with RCC die because of disease progression, making this cancer one of the most lethal malignant tumors. Thus, there is a great need for new treatment options for this cancer.

### SUMMARY OF THE INVENTION

This disclosure relates, in part, to methods of treating a subject with a RCC with a combination of lenvatinib or a pharmaceutically acceptable salt thereof and everolimus, wherein the dosage of one or both components of the combination treatment is modified upon the occurrence of one or more adverse events in the treated subject.

In a first aspect, the disclosure features a method of treating RCC. The method involves administering to a human subject that has a RCC a first dosage regimen comprising (i) lenvatinib or a pharmaceutically acceptable salt thereof at a dose of 18 mg/day and (ii) everolimus at a dose of 5 mg/day. In certain embodiments, following or during treatment with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 14 mg/day. In other embodiments, following or during treatment with the first dosage regimen, the human subject does not develop an adverse reaction, or develops an occurrence of a Grade 1 or tolerable Grade 2 adverse reaction. In such embodiments, the method further involves continuing administration of the first dosage regimen to the human subject (i.e., not lowering the dose of the first dosage regimen).

As used throughout this disclosure, a dosage regimen comprising lenvatinib or a pharmaceutically acceptable salt thereof at a specified dose means that lenvatinib or a pharmaceutically acceptable salt thereof is present in the dosage regimen at the specified dose. Although such a dosage regimen can contain additional components, lenvatinib or a pharmaceutically acceptable salt thereof is present only at the specific dose listed. Similarly, as used throughout this application, a dosage regimen comprising everolimus at a specified dose means that everolimus is present in the dosage regimen at the specified dose. Although such a dosage regimen can contain additional components, everolimus is present only at the specific dose listed. The dose of lenvatinib or a pharmaceutically acceptable salt thereof (e.g., 18 mg, 14 mg, 10 mg, 8 mg, or 6 mg) or everolimus (e.g., 5 mg or 2.5 mg) as used throughout refers to the dose of the free form of lenvatinib or everolimus, respectively.

In a second aspect, the disclosure provides a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. In carrying out this method, the human subject develops an occurrence of a first persistent and intolerable Grade 2 or Grade 3 adverse reaction or Grade 4 laboratory abnormality during or following treatment with the first dosage regimen. Thereupon, the method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 14 mg/day. In certain embodiments, following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 10 mg/day. In other embodiments, following or during treatment with the second dosage regimen, the human subject does not develop an adverse reaction, or develops an occurrence of a Grade 1 or tolerable Grade 2 adverse reaction. In such embodiments, the method further involves continuing administration of the second dosage regimen to the human subject (i.e., not lowering the dose being given in the second dosage regimen).

In a third aspect, the disclosure features a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 14 mg/day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 10 mg/day. In certain embodiments, following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising lenvatinib or a pharmaceutically acceptable salt thereof at a dose of 8 mg/day. In other embodiments, following or during treatment with the third dosage regimen, the human subject does not develop an adverse reaction, or develops an occurrence of a Grade 1 or tolerable Grade 2 adverse reaction. In such embodiments, the method further involves continuing administration of the third dosage regimen to the human subject (i.e., not lowering the dose being given in the third dosage regimen).

In a fourth aspect, the disclosure features a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 14 mg/day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 10 mg/day. Following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising lenvatinib or a pharmaceutically acceptable salt thereof at a dose of 8 mg/day. In certain embodiments, following or during treatment with the fourth dosage regimen, the human subject develops a fourth persistent and intolerable Grade 2 or Grade 3 adverse reaction or Grade 4 laboratory abnormality during treatment with the fourth dosage regimen. The method further involves discontinuing administration of the fourth dosage regimen to the human subject. In other embodiments, following or during treatment with the fourth dosage regimen, the human subject does not develop an adverse reaction, or develops an occurrence of a Grade 1 or tolerable Grade 2 adverse reaction. The method further involves continuing administration of the fourth dosage regimen to the human subject (i.e., not lowering the dose being given in the fourth dosage regimen).

The following embodiments apply to all of the above aspects. In certain embodiments, each of the second dosage regimen, the third dosage regimen, and the fourth dosage regimen is not initiated until resolution of an adverse reaction or toxicity associated with administration of everolimus.

In other embodiments, each of the second dosage regimen, the third dosage regimen, and the fourth dosage regimen comprises everolimus at a dose of 5 mg/day.

In some embodiments, each of the second dosage regimen, the third dosage regimen, and the fourth dosage regimen comprises everolimus at a dose of 5 mg every other day.

In some embodiments, the second dosage regimen comprises everolimus at a dose of 5 mg/day, the third dosage regimen comprises everolimus at a dose of 5 mg/day, and the fourth dosage regimen comprises either no everolimus or everolimus at a dose of 5 mg every other day.

In some embodiments, the second dosage regimen comprises everolimus at a dose of 5 mg/day, the third dosage regimen comprises everolimus at a dose of 5 mg every other day, and the fourth dosage regimen comprises either no everolimus or everolimus at a dose of 5 mg every other day.

In other embodiments, each of the second dosage regimen, the third dosage regimen, and the fourth dosage regimen comprises everolimus at a dose of 2.5 mg/day.

In some embodiments, each of the second dosage regimen, the third dosage regimen, and the fourth dosage regimen comprises everolimus at a dose of 2.5 mg every other day.

In some embodiments, the second dosage regimen does not include everolimus. In some embodiments, the third dosage regimen does not include everolimus. In some embodiments, the fourth dosage regimen does not include everolimus. In other embodiments, the second dosage regimen and the third dosage regimen does not include everolimus. In yet other embodiments, the second dosage regimen and the fourth dosage regimen does not include everolimus. In certain embodiments, the third dosage regimen and the fourth dosage regimen does not include everolimus. In other embodiments, each of the second dosage regimen, the third dosage regimen, and the fourth dosage regimen does not include everolimus.

In a fifth aspect, the disclosure provides a method of treating RCC. The method involves administering to a human subject that has a renal cell carcinoma and severe renal impairment or severe hepatic impairment, a first dosage regimen comprising (i) lenvatinib or a pharmaceutically acceptable salt thereof at a dose of 14 mg/day and (ii) everolimus at a dose of 2.5 mg/day. During or following treatment with the first dosage regimen, the human subject develops an occurrence of a first persistent and intolerable Grade 2 or Grade 3 adverse reaction or Grade 4 laboratory abnormality. The method involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day.

In a sixth aspect, the disclosure features a method of treating RCC. The method involves administering to a human subject that has a renal cell carcinoma and severe renal impairment or severe hepatic impairment, a first dosage regimen comprising (i) lenvatinib or a pharmaceutically acceptable salt thereof at a dose of 14 mg/day and (ii) everolimus at a dose of 2.5 mg/day. During or following treatment with the first dosage regimen, the human subject develops an occurrence of a first persistent and intolerable Grade 2 or Grade 3 adverse reaction or Grade 4 laboratory abnormality. The method involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. During or following treatment with the second dosage regimen, the human subject develops an occurrence of a second persistent and intolerable Grade 2 or Grade 3 adverse reaction or Grade 4 laboratory abnormality. The method further involves terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 8 mg/day.

In a seventh aspect, the disclosure provides a method of treating RCC. The method involves administering to a human subject that has a renal cell carcinoma and severe renal impairment or severe hepatic impairment, a first dosage regimen comprising (i) lenvatinib or a pharmaceutically acceptable salt thereof at a dose of 14 mg/day and (ii) everolimus at a dose of 2.5 mg/day. During or following treatment with the first dosage regimen, the human subject develops an occurrence of a first persistent and intolerable Grade 2 or Grade 3 adverse reaction or Grade 4 laboratory abnormality. The method involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. During or following treatment with the second dosage regimen, the human subject develops an occurrence of a second persistent and intolerable Grade 2 or Grade 3 adverse reaction or Grade 4 laboratory abnormality. The method further involves terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 8 mg/day. During or following treatment with the third dosage regimen, the human subject develops an occurrence of a third persistent and intolerable Grade 2 or Grade 3 adverse reaction or Grade 4 laboratory abnormality. The method further involves terminating administration of the third dosage regimen after the occurrence of the third persistent and intolerable Grade 2 or Grade 3 adverse reaction or Grade 4 laboratory abnormality and administering to the human subject a fourth dosage regimen comprising lenvatinib or a pharmaceutically acceptable salt thereof at a dose of 6 mg/day.

The following embodiments apply to all of the fifth, sixth, and seventh aspects above.

In one embodiment, the human subject has severe renal impairment and has a creatinine clearance [CLcr] less than 30 mL/min as calculated by the Cockroft-Gault equation.

In another embodiment, the human subject has severe hepatic impairment and has a liver disease classified in Child-Pugh class C.

In certain embodiments, each of the second dosage regimen, the third dosage regimen, and the fourth dosage regimen is not initiated until resolution of an adverse reaction or toxicity associated with administration of everolimus.

In some embodiments, the second dosage regimen comprises everolimus at a dose of 2.5 mg/day. In other embodiments, the third dosage regimen comprises everolimus at a dose of 2.5 mg/day. In yet other embodiments, the fourth dosage regimen comprises everolimus at a dose of 2.5 mg/day. In certain embodiments, the second dosage regimen and the third dosage regimen, comprises everolimus at a dose of 2.5 mg/day. In some embodiments, the second dosage regimen and the fourth dosage regimen comprises everolimus at a dose of 2.5 mg/day. In other embodiments, the third dosage regimen and the fourth dosage regimen comprises everolimus at a dose of 2.5 mg/day. In some embodiments, each of the second dosage regimen, the third dosage regimen, and the fourth dosage regimen comprises everolimus at a dose of 2.5 mg/day.

In some embodiments, the second dosage regimen comprises everolimus at a dose of 2.5 mg every other day. In other embodiments, the third dosage regimen comprises everolimus at a dose of 2.5 mg every other day. In yet other embodiments, the fourth dosage regimen comprises everolimus at a dose of 2.5 mg every other day. In certain embodiments, the second dosage regimen and the third dosage regimen, comprises everolimus at a dose of 2.5 mg every other day. In some embodiments, the second dosage regimen and the fourth dosage regimen comprises everolimus at a dose of 2.5 mg every other day. In other embodiments, the third dosage regimen and the fourth dosage regimen comprises everolimus at a dose of 2.5 mg every other day. In some embodiments, each of the second dosage regimen, the third dosage regimen, and the fourth dosage regimen comprises everolimus at a dose of 2.5 mg every other day.

In some embodiments, the second dosage regimen does not include everolimus. In some embodiments, the third dosage regimen does not include everolimus. In some embodiments, the fourth dosage regimen does not include everolimus. In other embodiments, the second dosage regimen and the third dosage regimen does not include everolimus. In yet other embodiments, the second dosage regimen and the fourth dosage regimen does not include everolimus. In certain embodiments, the third dosage regimen and the fourth dosage regimen does not include everolimus. In other embodiments, each of the second dosage regimen, the third dosage regimen, and the fourth dosage regimen does not include everolimus.

In an eighth aspect, the disclosure features a method of treating RCC. The method involves administering to a human subject that has a RCC a first dosage regimen comprising (i) lenvatinib or a pharmaceutically acceptable salt thereof at a dose of 18 mg/day and (ii) everolimus at a dose of 5 mg/day. Following or during treatment with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising (i) lenvatinib or a pharmaceutically acceptable salt thereof at a dose of 14 mg/day; and optionally (ii) everolimus at a dose of 5 mg/day, 5 mg every other day, 2.5 mg/day, or 2.5 mg every other day.

In a ninth aspect, the disclosure provides a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. In carrying out this method, the human subject develops an occurrence of a first persistent and intolerable Grade 2 or Grade 3 adverse reaction or Grade 4 laboratory abnormality during or following treatment with the first dosage regimen. Thereupon, the method further involves terminating administration of the first dosage regimen after the occurrence of the first persistent and intolerable Grade 2 or Grade 3 adverse reaction or Grade 4 laboratory abnormality and administering to the human subject a second dosage regimen comprising (i) lenvatinib or a pharmaceutically acceptable salt thereof at a dose of 14 mg/day; and (ii) optionally everolimus at a dose of 5 mg/day, 5 mg every other day, 2.5 mg/day, or 2.5 mg every other day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising (i) lenvatinib or a pharmaceutically acceptable salt thereof at a dose of 10 mg/day, and optionally (ii) everolimus at a dose of 5 mg/day, 5 mg every other day, 2.5 mg/day, or 2.5 mg every other day.

In a tenth aspect, the disclosure features a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising (i) lenvatinib or a pharmaceutically acceptable salt thereof at a dose of 14 mg/day, and optionally (ii) everolimus at a dose of 5 mg/day, 5 mg every other day, 2.5 mg/day, or 2.5 mg every other day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising (i) lenvatinib or a pharmaceutically acceptable salt thereof at a dose of 10 mg/day and optionally (ii) everolimus at a dose of 5 mg/day, 5 mg every other day, 2.5 mg/day, or 2.5 mg every other day. Following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising (i) lenvatinib or a pharmaceutically acceptable salt thereof at a dose of 8 mg/day, and optionally (ii) everolimus at a dose of 5 mg/day, 5 mg every other day, 2.5 mg/day, or 2.5 mg every other day.

In an eleventh aspect, the disclosure features a method of treating RCC that involves administering to a human subject that has a RCC a first dosage regimen comprising (i) lenvatinib or a pharmaceutically acceptable salt thereof at a dose of 18 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising (i) lenvatinib or a pharmaceutically acceptable salt thereof at a dose of 14 mg/day, and (ii) everolimus at a dose of 5 mg/day or 5 mg every other day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising (i) lenvatinib or a pharmaceutically acceptable salt thereof at a dose of 10 mg/day, and (ii) everolimus at a dose of 5 mg/day or 5 mg every other day. Following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising (i) lenvatinib or a pharmaceutically acceptable salt thereof at a dose of 8 mg/day, and (ii) everolimus at a dose of 5 mg/day or 5 mg every other day.

In a twelfth aspect, the disclosure features a method of treating RCC. The method involves administering to a human subject that has a RCC a first dosage regimen comprising (i) lenvatinib or a pharmaceutically acceptable salt thereof at a dose of 14 mg/day and (ii) everolimus at a dose of 5 mg/day. In certain embodiments, following or during treatment with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. In other embodiments, following or during treatment with the first dosage regimen, the human subject does not develop an adverse reaction, or develops an occurrence of a Grade 1 or tolerable Grade 2 adverse reaction. In such embodiments, the method further involves continuing administration of the first dosage regimen to the human subject (i.e., not lowering the dose of the first dosage regimen).

In a thirteenth aspect, the disclosure provides a method of treating RCC that involves administering to a human subject that has a RCC a first dosage regimen comprising (i) lenvatinib or a pharmaceutically acceptable salt thereof at a dose of 14 mg/day and (ii) everolimus at a dose of 5 mg/day. In carrying out this method, the human subject develops an occurrence of a first persistent and intolerable Grade 2 or Grade 3 adverse reaction or Grade 4 laboratory abnormality during or following treatment with the first dosage regimen. Thereupon, the method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. In certain embodiments, following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 8 mg/day. In other embodiments, following or during treatment with the second dosage regimen, the human subject does not develop an adverse reaction, or develops an occurrence of a Grade 1 or tolerable Grade 2 adverse reaction. In such embodiments, the method further involves continuing administration of the second dosage regimen to the human subject (i.e., not lowering the dose being given in the second dosage regimen).

In a fourteenth aspect, the disclosure features a method of treating RCC that involves administering to a human subject that has a RCC a first dosage regimen comprising (i) lenvatinib or a pharmaceutically acceptable salt thereof at a dose of 14 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 8 mg/day. In certain embodiments, following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising lenvatinib or a pharmaceutically acceptable salt thereof at a dose of 4 mg/day. In other embodiments, following or during treatment with the third dosage regimen, the human subject does not develop an adverse reaction, or develops an occurrence of a Grade 1 or tolerable Grade 2 adverse reaction. In such embodiments, the method further involves continuing administration of the third dosage regimen to the human subject (i.e., not lowering the dose being given in the third dosage regimen).

In a fifteenth aspect, the disclosure features a method of treating RCC that involves administering to a human subject that has a RCC a first dosage regimen comprising (i) lenvatinib or a pharmaceutically acceptable salt thereof at a dose of 14 mg/day and (ii) everolimus at a dose of 5 mg/day. Following or during therapy with the first dosage regimen, the human subject develops an occurrence of a first persistent and intolerable Grade 2 or Grade 3 adverse reaction or Grade 4 laboratory abnormality. The method further involves terminating administration of the first dosage regimen after the occurrence of the first persistent and intolerable Grade 2 or Grade 3 adverse reaction or Grade 4 laboratory abnormality and administering to the human subject a second dosage regimen comprising lenvatinib or a pharmaceutically acceptable salt thereof at a dose of 10 mg/day. Following or during treatment with the second dosage regimen, the human subject develops an occurrence of a second persistent and intolerable Grade 2 or Grade 3 adverse reaction or Grade 4 laboratory. The method further comprises terminating administration of the second dosage regimen after the occurrence of the second persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 8 mg/day. Following or during treatment with the third dosage regimen, the human subject develops an occurrence of a third persistent and intolerable Grade 2 or Grade 3 adverse reaction or Grade 4 laboratory abnormality during treatment with the third dosage regimen. The method further includes terminating administration of the third dosage regimen after the occurrence of the third persistent and intolerable Grade 2 or Grade 3 adverse reaction or Grade 4 laboratory abnormality and administering to the human subject a fourth dosage regimen comprising lenvatinib or a pharmaceutically acceptable salt thereof at a dose of 4 mg/day. In certain embodiments, following or during treatment with the fourth dosage regimen, the human subject develops a fourth persistent and intolerable Grade 2 or Grade 3 adverse reaction or Grade 4 laboratory abnormality during treatment with the fourth dosage regimen. In such embodiments, the method further involves discontinuing administration of the fourth dosage regimen to the human subject. In other embodiments, following or during treatment with the fourth dosage regimen, the human subject does not develop an adverse reaction, or develops an occurrence of a Grade 1 or tolerable Grade 2 adverse reaction. In such embodiments, the method further involves continuing administration of the fourth dosage regimen to the human subject (i.e., not lowering the dose being given in the fourth dosage regimen).

The following embodiments apply to the twelfth, thirteenth, fourteenth, and fifteenth aspects.

In one embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline. In one embodiment, the third dosage regimen is not initiated until the second persistent and intolerable Grade 2 or Grade 3 adverse reaction or Grade 4 laboratory abnormality is resolved to tolerable Grade 2, Grade 0-1, or baseline. In one embodiment, the fourth dosage regimen is not initiated until the third persistent and intolerable Grade 2 or Grade 3 adverse reaction or Grade 4 laboratory abnormality is resolved to tolerable Grade 2, Grade 0-1, or baseline. In another embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline and the third dosage regimen is not initiated until the second persistent and intolerable Grade 2 or Grade 3 adverse reaction or Grade 4 laboratory abnormality is resolved to tolerable Grade 2, Grade 0-1, or baseline. In another embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline; the third dosage regimen is not initiated until the second persistent and intolerable Grade 2 or Grade 3 adverse reaction or Grade 4 laboratory abnormality is resolved to tolerable Grade 2, Grade 0-1, or baseline; and the fourth dosage regimen is not initiated until the third persistent and intolerable Grade 2 or Grade 3 adverse reaction or Grade 4 laboratory abnormality is resolved to tolerable Grade 2, Grade 0-1, or baseline.

In some embodiments, medical management of each of the first, second, and third persistent and intolerable Grade 2 or Grade 3 adverse reactions or Grade 4 laboratory abnormalities is initiated prior to terminating administration of the dosage regimen administered at the time of onset of the adverse reaction or laboratory abnormality.

In certain embodiments, medical management of each of the first, second, and third persistent and intolerable Grade 2 or Grade 3 adverse reactions or Grade 4 laboratory abnormalities is initiated prior to initiating administration of the dosage regimen that occurs after resolution of the adverse reaction or laboratory abnormality to tolerable Grade 2, Grade 0-1, or baseline.

In some embodiments, the first persistent and intolerable Grade 2 or Grade 3 adverse reaction or Grade 4 laboratory abnormality is the same as the second and/or third persistent and intolerable Grade 2 or Grade 3 adverse reaction or Grade 4 laboratory abnormality.

In certain embodiments, the first persistent and intolerable Grade 2 or Grade 3 adverse reaction or Grade 4 laboratory abnormality is different from the second and/or third persistent and intolerable Grade 2 or Grade 3 adverse reaction or Grade 4 laboratory abnormality.

In some embodiments, the Grade 2 or Grade 3 adverse reaction is selected from the group consisting of Grade 3 hypertension, Grade 2 hypertension, Grade 3 cardiac dysfunction, Grade 2 cardiac dysfunction, Grade 3 arterial thromboembolic event, Grade 2 arterial thromboembolic event, Grade 3 proteinuria, Grade 2 proteinuria, Grade 3 renal failure or impairment, Grade 2 renal failure or impairment, Grade 3 diarrhea, Grade 2 diarrhea, Grade 3 gastrointestinal perforation or fistula, Grade 2 gastrointestinal perforation or fistula, Grade 3 vomiting, Grade 2 vomiting, Grade 3 decreased appetite, Grade 2 decreased appetite, Grade 3 fatigue, Grade 2 fatigue, Grade 3 nausea, Grade 2 nausea, Grade 3 cough, Grade 2 cough, Grade 3 decreased weight, Grade 2 decreased weight, Grade 3 dehydration, Grade 2 dehydration, Grade 3 thrombocytopenia, Grade 2 thrombocytopenia, Grade 3 anemia, Grade 2 anemia, Grade 3 acute renal failure, Grade 2 acute renal failure, Grade 3 QT/QTc interval prolongation, Grade 2 QT/QTc interval prolongation, Grade 3 reversible posterior leukoencephalopathy syndrome (RPLS), Grade 2 RPLS, Grade 3 hemorrhagic events, Grade 2 hemorrhagic events, Grade 3 hyperthyroidism, and Grade 2 hyperthyroidism. In some instances, the Grade 2 or Grade 3 adverse reaction is selected from the group consisting of Grade 3 diarrhea, Grade 2 diarrhea, Grade 3 vomiting, Grade 2 vomiting, Grade 3 nausea, Grade 2 nausea, Grade 3 proteinuria, and Grade 2 proteinuria.

In certain embodiments, the Grade 4 laboratory abnormality is selected from the group consisting of Grade 4 increase in aspartate aminotransferase, Grade 4 increase in alanine aminotransferase, Grade 4 increase in alkaline phosphatase, Grade 4 hyperkalemia, Grade 4 hypokalemia, Grade 4 hyponatremia, Grade 4 hypocalcemia, Grade 4 hypophosphatemia, Grade 4 hyperglycemia, Grade 4 hypertriglyceridemia, Grade 4 increase in cholesterol, Grade 4 increase in lipase, Grade 4 decrease in hemoglobin, Grade 4 decrease in platelet count, and Grade 4 decrease in lymphocyte count. In some instances, the Grade 4 laboratory abnormality is selected from the group consisting of Grade 4 increase in lipase, Grade 4 hypertriglyceridemia, Grade 4 increase in cholesterol, Grade 4 hypophosphatemia, Grade 4 hyponatremia, and Grade 4 hypokalemia.

In certain embodiments, each of the second dosage regimen, the third dosage regimen, and the fourth dosage regimen is not initiated until resolution of an adverse reaction or toxicity associated with administration of everolimus.

In other embodiments, each of the second dosage regimen, the third dosage regimen, and the fourth dosage regimen comprises everolimus at a dose of 5 mg/day.

In some embodiments, each of the second dosage regimen, the third dosage regimen, and the fourth dosage regimen comprises everolimus at a dose of 5 mg every other day.

In some embodiments, the second dosage regimen comprises everolimus at a dose of 5 mg/day, the third dosage regimen comprises everolimus at a dose of 5 mg/day, and the fourth dosage regimen comprises either no everolimus or everolimus at a dose of 5 mg every other day.

In some embodiments, the second dosage regimen comprises everolimus at a dose of 5 mg/day, the third dosage regimen comprises everolimus at a dose of 5 mg every other day, and the fourth dosage regimen comprises either no everolimus or everolimus at a dose of 5 mg every other day.

In some embodiments, the second dosage regimen does not include everolimus. In some embodiments, the third dosage regimen does not include everolimus. In some embodiments, the fourth dosage regimen does not include everolimus. In other embodiments, the second dosage regimen and the third dosage regimen does not include everolimus. In yet other embodiments, the second dosage regimen and the fourth dosage regimen does not include everolimus. In certain embodiments, the third dosage regimen and the fourth dosage regimen does not include everolimus. In other embodiments, each of the second dosage regimen, the third dosage regimen, and the fourth dosage regimen does not include everolimus.

In a sixteenth aspect, the disclosure provides a method of treating renal cell carcinoma in a human subject in need thereof. The method involves administering to a human subject that has a renal cell carcinoma a first dosage regimen comprising (i) lenvatinib or a pharmaceutically acceptable salt thereof at a dose of 14 mg/day and (ii) everolimus at a dose of 5 mg/day for a treatment period, wherein the human subject does not develop an intolerable Grade 2 or Grade 3 adverse reaction or Grade 4 laboratory abnormality during the treatment period with the first dosage regimen. The method further involves terminating administration of the first dosage regimen after the treatment period and administering to the human subject a second dosage regimen comprising (i) lenvatinib or a pharmaceutically acceptable salt thereof at a dose of 18 mg/day and (ii) everolimus at a dose of 5 mg/day.

In some embodiments of this aspect, the human subject develops an occurrence of a first persistent and intolerable Grade 2 or Grade 3 adverse reaction or Grade 4 laboratory abnormality during treatment with the second dosage regimen. The method further comprises terminating administration of the second dosage regimen after the occurrence of the first persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 14 mg/day. In some instances, the human subject develops an occurrence of a second persistent and intolerable Grade 2 or Grade 3 adverse reaction or Grade 4 laboratory abnormality during treatment with the third dosage regimen. In such cases, the administration of the third dosage regimen is terminated and the human subject is administered a fourth dosage regimen comprising lenvatinib or a pharmaceutically acceptable salt thereof at a dose of 10 mg/day. In other instances, the human subject does not develop an adverse reaction, or develops a Grade 1 or tolerable Grade 2 adverse reaction. In such instances, the human subject can continue being administered the third dosage regimen.

In some embodiments of the sixteenth aspect, the human subject develops an occurrence of a first persistent and intolerable Grade 2 or Grade 3 adverse reaction or Grade 4 laboratory abnormality during treatment with the second dosage regimen. The method further comprises terminating administration of the second dosage regimen after the occurrence of the first persistent and intolerable Grade 2 or Grade 3 adverse reaction or Grade 4 laboratory abnormality and administering to the human subject a third dosage regimen comprising lenvatinib or a pharmaceutically acceptable salt thereof at a dose of 14 mg/day. The human subject develops an occurrence of a second persistent and intolerable Grade 2 or Grade 3 adverse reaction or Grade 4 laboratory abnormality during treatment with the third dosage regimen and the administration of the third dosage regimen is terminated and the human subject is administered a fourth dosage regimen comprising lenvatinib or a pharmaceutically acceptable salt thereof at a dose of 10 mg/day. In certain instances, the human subject develops an occurrence of a third persistent and intolerable Grade 2 or Grade 3 adverse reaction or Grade 4 laboratory abnormality during treatment with the fourth dosage regimen and the administration of the fourth dosage regimen is terminated and the human subject is administered a fifth dosage regimen comprising lenvatinib or a pharmaceutically acceptable salt thereof at a dose of 8 mg/day. In other instances, the human subject does not develop an adverse reaction, or develops a Grade 1 or tolerable Grade 2 adverse reaction. In such instances, the human subject can continue being administered the fourth dosage regimen.

In certain embodiments of this aspect, the treatment period with the first dosage regimen comprises 28 days.

In certain embodiments of this aspect, the treatment period with the first dosage regimen consists of 28 days.

The following embodiments apply to all of the above aspects described above.

In one embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline. In one embodiment, the third dosage regimen is not initiated until the second persistent and intolerable Grade 2 or Grade 3 adverse reaction or Grade 4 laboratory abnormality is resolved to tolerable Grade 2, Grade 0-1, or baseline. In one embodiment, the fourth dosage regimen is not initiated until the third persistent and intolerable Grade 2 or Grade 3 adverse reaction or Grade 4 laboratory abnormality is resolved to tolerable Grade 2, Grade 0-1, or baseline. In another embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline and the third dosage regimen is not initiated until the second persistent and intolerable Grade 2 or Grade 3 adverse reaction or Grade 4 laboratory abnormality is resolved to tolerable Grade 2, Grade 0-1, or baseline. In another embodiment, the second dosage regimen is not initiated until the first persistent and intolerable Grade 2 or Grade 3 adverse reaction or Grade 4 laboratory abnormality is resolved to tolerable Grade 2, Grade 0-1, or baseline; the third dosage regimen is not initiated until the second persistent and intolerable Grade 2 or Grade 3 adverse reaction or Grade 4 laboratory abnormality is resolved to tolerable Grade 2, Grade 0-1, or baseline; and the fourth dosage regimen is not initiated until the third persistent and intolerable Grade 2 or Grade 3 adverse reaction or Grade 4 laboratory abnormality is resolved to tolerable Grade 2, Grade 0-1, or baseline.

In one embodiment, the second dosage regimen is not initiated until the first persistent and intolerable Grade 2 or Grade 3 adverse reaction or Grade 4 laboratory abnormality is resolved to Grade 0-1 or tolerable Grade 2. In one embodiment, the third dosage regimen is not initiated until the second persistent and intolerable Grade 2 or Grade 3 adverse reaction or Grade 4 laboratory abnormality is resolved to Grade 0-1 or tolerable Grade 2. In one embodiment, the fourth dosage regimen is not initiated until the third persistent and intolerable Grade 2 or Grade 3 adverse reaction or Grade 4 laboratory abnormality is resolved to Grade 0-1 or tolerable Grade 2. In another embodiment, the second dosage regimen is not initiated until the first persistent and intolerable Grade 2 or Grade 3 adverse reaction or Grade 4 laboratory abnormality is resolved to Grade 0-1 or tolerable Grade 2 and the third dosage regimen is not initiated until the second persistent and intolerable Grade 2 or Grade 3 adverse reaction or Grade 4 laboratory abnormality is resolved to Grade 0-1 or tolerable Grade 2. In another embodiment, the second dosage regimen is not initiated until the first persistent and intolerable Grade 2 or Grade 3 adverse reaction or Grade 4 laboratory abnormality is resolved to Grade 0-1 or tolerable Grade 2; the third dosage regimen is not initiated until the second persistent and intolerable Grade 2 or Grade 3 adverse reaction or Grade 4 laboratory abnormality is resolved to Grade 0-1 or tolerable Grade 2; and the fourth dosage regimen is not initiated until the third persistent and intolerable Grade 2 or Grade 3 adverse reaction or Grade 4 laboratory abnormality is resolved to Grade 0-1 or tolerable Grade 2.

In some embodiments, medical management of each of the first, second, and third persistent and intolerable Grade 2 or Grade 3 adverse reactions or Grade 4 laboratory abnormalities is initiated prior to terminating administration of the dosage regimen administered at the time of onset of the adverse reaction or laboratory abnormality.

In certain embodiments, medical management of each of the first, second, and third persistent and intolerable Grade 2 or Grade 3 adverse reactions or Grade 4 laboratory abnormalities is initiated prior to initiating administration of the dosage regimen that occurs after resolution of the adverse reaction or laboratory abnormality to tolerable Grade 2, Grade 0-1, or baseline.

In certain embodiments, medical management of each of the first, second, and third persistent and intolerable Grade 2 or Grade 3 adverse reactions or Grade 4 laboratory abnormalities is initiated prior to initiating administration of the dosage regimen that occurs after resolution of the adverse reaction or laboratory abnormality to Grade 0-1 or tolerable Grade 2.

In some embodiments, the first persistent and intolerable Grade 2 or Grade 3 adverse reaction or Grade 4 laboratory abnormality is the same as the second and/or third persistent and intolerable Grade 2 or Grade 3 adverse reaction or Grade 4 laboratory abnormality.

In certain embodiments, the first persistent and intolerable Grade 2 or Grade 3 adverse reaction or Grade 4 laboratory abnormality is different from the second and/or third persistent and intolerable Grade 2 or Grade 3 adverse reaction or Grade 4 laboratory abnormality.

In some embodiments, the Grade 2 or Grade 3 adverse reaction is selected from the group consisting of Grade 3 hypertension, Grade 2 hypertension, Grade 3 cardiac dysfunction, Grade 2 cardiac dysfunction, Grade 3 arterial thromboembolic event, Grade 2 arterial thromboembolic event, Grade 3 proteinuria, Grade 2 proteinuria, Grade 3 renal failure or impairment, Grade 2 renal failure or impairment, Grade 3 diarrhea, Grade 2 diarrhea, Grade 3 gastrointestinal perforation or fistula, Grade 2 gastrointestinal perforation or fistula, Grade 3 vomiting, Grade 2 vomiting, Grade 3 decreased appetite, Grade 2 decreased appetite, Grade 3 fatigue, Grade 2 fatigue, Grade 3 nausea, Grade 2 nausea, Grade 3 cough, Grade 2 cough, Grade 3 decreased weight, Grade 2 decreased weight, Grade 3 dehydration, Grade 2 dehydration, Grade 3 thrombocytopenia, Grade 2 thrombocytopenia, Grade 3 anemia, Grade 2 anemia, Grade 3 acute renal failure, Grade 2 acute renal failure, Grade 3 QT/QTc interval prolongation, Grade 2 QT/QTc interval prolongation, Grade 3 reversible posterior leukoencephalopathy syndrome (RPLS), Grade 2 RPLS, Grade 3 hemorrhagic events, Grade 2 hemorrhagic events, Grade 3 hyperthyroidism, and Grade 2 hyperthyroidism. In some instances, the Grade 2 or Grade 3 adverse reaction is selected from the group consisting of Grade 3 diarrhea, Grade 2 diarrhea, Grade 3 vomiting, Grade 2 vomiting, Grade 3 nausea, Grade 2 nausea, Grade 3 proteinuria, and Grade 2 proteinuria.

In certain embodiments, the Grade 4 laboratory abnormality is selected from the group consisting of Grade 4 increase in aspartate aminotransferase, Grade 4 increase in alanine aminotransferase, Grade 4 increase in alkaline phosphatase, Grade 4 hyperkalemia, Grade 4 hypokalemia, Grade 4 hyponatremia, Grade 4 hypocalcemia, Grade 4 hypophosphatemia, Grade 4 hyperglycemia, Grade 4 hypertriglyceridemia, Grade 4 increase in cholesterol, Grade 4 increase in lipase, Grade 4 decrease in hemoglobin, Grade 4 decrease in platelet count, and Grade 4 decrease in lymphocyte count. In some instances, the Grade 4 laboratory abnormality is selected from the group consisting of Grade 4 increase in lipase, Grade 4 hypertriglyceridemia, Grade 4 increase in cholesterol, Grade 4 hypophosphatemia, Grade 4 hyponatremia, and Grade 4 hypokalemia.

In certain embodiments, each of the second dosage regimen, the third dosage regimen, and the fourth dosage regimen is not initiated until resolution of an adverse reaction or toxicity associated with administration of everolimus.

In some embodiments, the second dosage regimen does not include everolimus. In some embodiments, the third dosage regimen does not include everolimus. In some embodiments, the fourth dosage regimen does not include everolimus. In other embodiments, the second dosage regimen and the third dosage regimen does not include everolimus. In yet other embodiments, the second dosage regimen and the fourth dosage regimen does not include everolimus. In certain embodiments, the third dosage regimen and the fourth dosage regimen does not include everolimus. In other embodiments, each of the second dosage regimen, the third dosage regimen, and the fourth dosage regimen does not include everolimus.

In certain embodiments, lenvatinib or the pharmaceutically acceptable salt thereof is formulated as a capsule.

In some embodiments, everolimus is formulated as a tablet.

In certain embodiments, lenvatinib or the pharmaceutically acceptable salt thereof and everolimus are administered to the human subject orally.

In some embodiments, the human subject has received a prior vascular endothelial growth factor (VEGF)-targeted therapy.

In some embodiments, lenvatinib or the pharmaceutically acceptable salt thereof and everolimus are administered once daily.

In certain embodiments, lenvatinib or the pharmaceutically acceptable salt thereof and everolimus are administered once daily for at least 28 weeks, at least 56 weeks, at least 84 weeks, at least 112 weeks, at least 140 weeks, or at least 168 weeks.

In some embodiments, the renal cell carcinoma is an unresectable advanced renal cell carcinoma.

In other embodiments, the renal cell carcinoma is a metastatic renal cell carcinoma.

In yet other embodiments, the renal cell carcinoma is an advanced renal cell carcinoma (e.g., advanced renal cell carcinoma following a prior anti-angiogenic therapy). In certain embodiments, the prior anti-angiogenic therapy is a VEGF-targeted therapy.

In some embodiments, lenvatinib or a pharmaceutically acceptable salt thereof is lenvatinib mesylate.

In certain embodiments, the human subject has a poor MSKCC risk score.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description and from the claims.

### BRIEF DESCRIPTION OF DRAWINGS

FIG. 1 is a Kaplan-Meier plot of Progression-Free Survival comparing the three arms of the study. Arm A=LENVIMA® 18 mg+Everolimus 5 mg (top line in figure); Arm B=LENVIMA® 24 mg (middle line in figure); and Arm C=Everolimus 10 mg (bottom line in figure). Hazard ratio is based on a stratified Cox regression model including treatment as a factor and hemoglobin and corrected serum calcium as strata. The Efron method was used for correction of tied events. Median survival is based on Kaplan-Meier method and 95% confidence interval is based on the Greenwood formula using log-log transformation.

FIG. 2 is a Kaplan-Meier plot of Overall Survival comparing the three arms of the study. Arm A=LENVIMA® 18 mg+Everolimus 5 mg (top line in figure); Arm B=LENVIMA® 24 mg (middle line in figure); and Arm C=Everolimus 10 mg (bottom line in figure). Hazard ratio is based on a stratified Cox regression model including treatment as a factor and hemoglobin and corrected serum calcium as strata. The Efron method was used for correction of tied events. Median survival is based on Kaplan-Meier method and 95% confidence interval is based on the Greenwood formula using log-log transformation.

FIG. 3 is a Kaplan-Meier plot of Overall Survival comparing the three arms of the study. Arm A=LENVIMA® 18 mg+Everolimus 5 mg (top line in figure); Arm B=LENVIMA® 24 mg (middle line in figure); and Arm C=Everolimus 10 mg (bottom line in figure). Hazard ratio is based on a stratified Cox regression model including treatment as a factor and hemoglobin and corrected serum calcium as strata. The Efron method was used for correction of tied events. Median survival is based on Kaplan-Meier method and 95% confidence interval is based on the Greenwood formula using log-log transformation. The data cut-off date was Jul. 31, 2015.

FIG. 4 is a Kaplan-Meier plot of Progression-Free Survival based on Investigator Assessment comparing two arms of the study. Arm A=LENVIMA® 18 mg+Everolimus 5 mg (top line in figure) and Arm B=LENVIMA® 14 mg+Everolimus 5 mg (bottom line in figure). Median survival is based on Kaplan-Meier method and 95% confidence interval is based on the Greenwood formula using log-log transformation.

FIG. 5 is a Kaplan-Meier plot of Progression-Free Survival based on IIR Assessment comparing two arms of the study. Arm A=LENVIMA® 18 mg+Everolimus 5 mg (top line in figure) and Arm B=LENVIMA® 14 mg+Everolimus 5 mg (bottom line in figure). Median survival is based on Kaplan-Meier method and 95% confidence interval is based on the Greenwood formula using log-log transformation.

FIG. 6 illustrates the investigator assessment of the percentage changes in the sum of diameters of target lesions from two arms of the study. Arm A=LENVIMA® 18 mg+Everolimus 5 mg (right figure) and Arm B=LENVIMA® 14 mg+Everolimus 5 mg (left figure).

FIG. 7 is a forest plot depicting the investigator assessments of the subgroup analysis of the objective response rates for two arms of the study. Arm A=LENVIMA® 18 mg+Everolimus 5 mg (favor for arm is to the left of line) and Arm B=LENVIMA® 14 mg+Everolimus 5 mg (favor for arm is to the right of the line).

FIG. 8 is a Kaplan-Meier plot comparing the Overall Survival rates of two arms of the study. Arm A=LENVIMA® 18 mg+Everolimus 5 mg (top line in figure) and Arm B=LENVIMA® 14 mg+Everolimus 5 mg (bottom line in figure). Median survival is based on Kaplan-Meier method and 95% confidence interval is based on the Greenwood formula using log-log transformation.

FIG. 9 is a forest plot depicting the subgroup analysis of subjects with intolerable grade 2 or any ≥grade 3 treatment-emergent adverse events within 24 weeks after randomization for two arms of the study. Arm A=LENVIMA® 18 mg+Everolimus 5 mg (favor for arm is to the left of line) and Arm B=LENVIMA® 14 mg+Everolimus 5 mg (favor for arm is to the right of the line).

### DESCRIPTION OF EMBODIMENTS

This application provides methods of treating a human subject that has a renal cell carcinoma (e.g., advanced RCC, unresectable advanced RCC, or metastatic RCC). The method involves administering to the subject a combination of everolimus (5 mg) and lenvatinib or a pharmaceutically acceptable salt thereof (18 mg or 14 mg as a starting dose (also a starting dose of 14 mg or 10 mg if the subject has severe renal or hepatic impairment)). If the subject develops one or more adverse events as a result of the treatment with lenvatinib or a pharmaceutically acceptable salt thereof and/or everolimus, the application provides modifications of the treatment regimen as well as adjusted dosing regimens (reduced doses of one or both lenvatinib and everolimus). If the subject does not develop an adverse reaction as a result of administration of a starting dose of 14 mg of lenvatinib or a pharmaceutically acceptable salt thereof in combination with 5 mg of everolimus, the subject can be up-titrated to a higher dosage regimen (e,g., 18 mg of lenvatinib or a pharmaceutically acceptable salt thereof in combination with 5 mg of everolimus).

Renal Cell Carcinoma

Cancers of the kidney account for about 2.5% of the total human cancer burden, with approximately 338,000 new cases diagnosed in 2012. They occur in all world regions, with a predominance in developed countries. There are several types of kidney cancer of which renal cell carcinoma (RCC) is the most common (over 90%). RCC arises in the cells of the proximal renal tubular epithelium of the renal tubules and is often asymptomatic, being detected incidentally at imaging investigations when a person is being examined for other ailments. Hematuria, pain, and flank mass are the classic triad of presenting symptoms, but a large percentage of patients lack all of these symptoms and present instead with systemic symptoms including chronic fatigue, weight loss, abdominal pain, abdominal mass, anorexia, anemia, hypercalcemia, sleep disturbances, and recurrent fevers. This cancer shows a clear predominance in men, with men representing two-thirds of cases. Approximately 40% of patients with RCC die because of disease progression.

According to the 2004 World Health Organization classification, several histological RCC subtypes are recognized including: clear cell RCC, multiocular clear cell RCC, papillary RCC, and chromophobe RCC, carcinoma of the collecting ducts of Bellini, renal medullary carcinoma, Xp11 translocation carcinomas, carcinoma associated with neuroblastoma, mucinous tubular and spindle cell carcinoma, papillary adenoma, oncocytomas, and renal cell carcinoma unclassified. In 2013, the classification working group of the International Society of Urological Pathology (ISUP) consensus conference on renal neoplasia suggested the addition of five new well-characterized types of renal neoplasms as new distinct epithelial tumors within the classification system: tubulocystic RCC, acquired cystic disease-associated RCC, clear cell (tubulo) papillary RCC, the MiT family translocation RCCs (in particular t(6;11) RCC), and hereditary leiomyomatosis RCC syndrome-associated RCC. In addition, the ISUP also suggested three additional types considered as new and emerging entities: thyroid-like follicular RCC; succinate dehydrogenase B deficiency-associated RCC; and ALK translocation RCC. Of the several histological RCC subtypes, clear cell RCC, papillary RCC, and chromophobe RCC are the most frequent histological subtypes, together accounting for more than 90% of all RCCs.

The staging of RCC is important for determining how best to treat the disease. RCC can be staged using the TNM staging system, where the size and extent of the tumor (T), involvement of lymph nodes (N) and metastases (M) are classified separately. Also, it can use overall stage grouping into stage I-IV, with the 1997 revision of AJCC described below:

Stage I: Tumor of a diameter of 7 cm (approx. 2¾ inches) or smaller, and limited to the kidney. No lymph node involvement or metastases to distant organs.

Stage II: Tumor larger than 7 cm but still limited to the kidney. No lymph node involvement or metastases to distant organs.

Stage III (either of the following): (1) Tumor of any size with involvement of a nearby lymph node but no metastases to distant organs. Tumor of this stage may be with or without spread to fatty tissue around the kidney, with or without spread into the large veins leading from the kidney to the heart. (2) Tumor with spread to fatty tissue around the kidney and/or spread into the large veins leading from the kidney to the heart, but without spread to any lymph nodes or other organs.

Stage IV (any of the following): (1) Tumor that has spread directly through the fatty tissue and the fascia ligament-like tissue that surrounds the kidney. (2) Involvement of more than one lymph node near the kidney. (3) Involvement of any lymph node not near the kidney. (4) Distant metastases, such as in the lungs, bone, or brain.

In certain embodiments, the RCC is an advanced RCC (e.g., advanced renal cell carcinoma following a prior anti-angiogenic therapy). In certain instances, the prior anti-angiogenic therapy is VEGF-targeted therapy. In other embodiments, the RCC is an unresectable advanced RCC. In yet other embodiments, the RCC is a metastatic RCC.

Treatment of RCC can involve surgery to remove part or all of the kidney (partial nephrectomy or nephrectomy). Surgery is most useful if the cancer is only in the kidneys. In the case of metastatic disease, surgical treatment may be an option depending on the stage of growth of the tumor and how far the disease has spread. If surgery is not a good option for the patient, percutaneous ablation therapies may be used such as radio frequency ablation and cryoablation. Another approach to treatment of RCC is to use immunotherapy to activate the patient's immune system to attack the cancer. A further approach is to use targeted therapies that target growth factors known to promote the growth and spread of tumors. These therapies include nivolumab, axitinib, sunitinib, bevacizumab, sorafenib, pazopanib, interferon-α, temsirolimus, cabozantinib, everolimus, and lenvatinib.

This disclosure provides methods of treating different types of RCC (e.g., those noted above) using a combination of everolimus and lenvatinib or a pharmaceutically acceptable salt thereof.

Lenvatinib

A number of kinase inhibitors have been developed as antitumor agents. For example, a group of compounds having inhibitory activity against receptor tyrosine kinases, such as vascular endothelial growth factor receptor (VEGFR), are known to inhibit angiogenesis and are regarded as a new class of antitumor agents. Lenvatinib is a multi-target receptor tyrosine kinase inhibitor that inhibits the kinase activities of VEGFR1 (FLT1), VEGFR2 (KDR), and VEGFR3 (FLT4). Lenvatinib also inhibits other receptor tyrosine kinases that have been implicated in pathogenic angiogenesis, tumor growth, and cancer progression in addition to their normal cellular functions, including fibroblast growth factor (FGF) receptors FGFR1, FGFR2, FGFR3, and FGFR4; rearranged during transfection receptor (RET), KIT, and platelet-derived growth factor receptor alpha (PDGFRα).

The term “lenvatinib” refers to 4-(3-chloro-4(cyclopropylaminocarbonyl)aminophenoxy)-7-methoxy-6-quinolinecarboxamide. This compound is disclosed in Example 368 (see, column 270) of U.S. Pat. No. 7,253,286. U.S. Pat. No. 7,253,286 is incorporated by reference in its entirety herein. The term “pharmaceutically acceptable salt” is not particularly restricted as to the type of salt. Examples of such salts include, but are not limited to, inorganic acid addition salt such as hydrochloric acid salt, sulfuric acid salt, carbonic acid salt, bicarbonate salt, hydrobromic acid salt, and hydriodic acid salt; organic carboxylic acid addition salt such as acetic acid salt, maleic acid salt, lactic acid salt, tartaric acid salt, and trifluoroacetic acid salt; organic sulfonic acid addition salt such as methanesulfonic acid salt, hydroxymethanesulfonic acid salt, hydroxyethanesulfonic acid salt, benzenesulfonic acid salt, toluenesulfonic acid salt, and taurine salt; amine addition salt such as trimethylamine salt, triethylamine salt, pyridine salt, procaine salt, picoline salt, dicyclohexylamine salt, N,N′-dibenzylethylenediamine salt, N-methylglucamine salt, diethanolamine salt, triethanolamine salt, tris(hydroxymethylamino)methane salt, and phenethylbenzylamine salt; and amino acid addition salt such as arginine salt, lysine salt, serine salt, glycine salt, aspartic acid salt, and glutamic acid salt. In one embodiment, the pharmaceutically acceptable salt is a methanesulfonic acid salt (“mesylate”). The methanesulfonic acid salt form (i.e., the mesylate) of lenvatinib is disclosed in U.S. Pat. No. 7,612,208, which is incorporated by reference herein in its entirety. The chemical name of lenvatinib mesylate is 4-[3-chloro-4-(N′-cyclopropylureido)phenoxy]-7-methoxyquinoline-6-carboxamide methanesulfonate and it chemical structure is provided below:

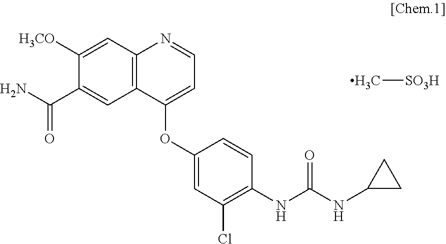

Lenvatinib mesylate is also referred to as LENVIMA®.

Lenvatinib mesylate is a white to pale reddish yellow powder. It is slightly soluble in water and practically insoluble in ethanol (dehydrated). The dissociation constant (pKa value) of lenvatinib mesylate is 5.05 at 25° C. The partition coefficient (log P value) is 3.30.

Everolimus

Everolimus is an inhibitor of mammalian target of rapamycin (mTOR), a serine-threonine kinase, downstream of the PI3K/AKT pathway. The mTOR pathway is dysregulated in several human cancers. Everolimus binds to an intracellular protein, FKBP-12, resulting in an inhibitory complex formation with mTOR complex 1 (mTORC1) and thus inhibition of mTOR kinase activity. Everolimus can reduce the activity of S6 ribosomal protein kinase (S6K1) and eukaryotic initiation factor 4E-binding protein (4E-BP1), downstream effectors of mTOR, involved in protein synthesis. S6K1 is a substrate of mTORC1 and phosphorylates the activation domain 1 of the estrogen receptor which results in ligand-independent activation of the receptor. In addition, everolimus can inhibit the expression of hypoxia-inducible factor (e.g., HIF-1) and reduce the expression of vascular endothelial growth factor (VEGF). Inhibition of mTOR by everolimus has been shown to reduce cell proliferation, angiogenesis, and glucose uptake in in vitro and/or in vivo studies.

The chemical name of everolimus is (1R,9S,12S,15R,16E,18R,19R,21R,23 S,24E,26E,28E,30S,32S,35R)-1,18-dihydroxy-12-{(1R)-2-[(1 S,3R,4R)-4-(2-hydroxyethoxy)-3-methoxycyclohexyl]-1-methyl ethyl}-19,30-dimethoxy-15,17,21,23,29,35-hexamethyl-11,36-dioxa-4-aza-tricyclo[30.3.1.0^{4,9}]hexatriaconta-16,24,26,28-tetraene-2,3,10,14,20-pentaone.

The structural formula of everolimus is:

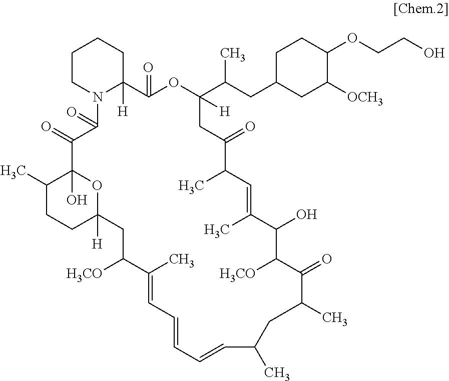 [SMILES: COC1CC2CCC(C)C(O)(O2)C(=O)C(=O)N2CCCCC2C(=O)OC(C(C)CC2CCC(OCCO)C(OC)C2)CC(=O)C(C)/C=C(\C)C(O)C(OC)C(=O)C(C)CC(C)/C=C/C=C/C=C/1C]

Everolimus is marketed under the tradename AFINITOR®. AFINITOR® tablets are supplied for oral administration and contain 2.5 mg, 5 mg, 7.5 mg, or 10 mg of everolimus. The tablets also contain anhydrous lactose, butylated hydroxytoluene, crospovidone, hypromellose, lactose monohydrate, and magnesium stearate as inactive ingredients.

Everolimus is indicated, inter alia, for the treatment of adults with advanced RCC after failure of treatment with sunitinib or sorafenib.

Combination Therapy for Treatment of RCC

The present disclosure provides, in part, a combination therapy for treatment of a human subject with RCC. In certain instances, the human subject has advanced RCC following one prior anti-angiogenic therapy. In a particular embodiment, the subject to be administered the combination therapy has had at least one prior VEGF-targeted treatment (e.g., sunitinib, pazopanib, tivozanib, axitinib, sorafenib, or bevacizumab). In other words, the combination therapy can be employed as a second-line therapy. In certain instances, the best response for the at least one prior VEGF-targeted therapy was a complete response. In certain instances, the best response for the at least one prior VEGF-targeted therapy was a partial response. In certain instances, the best response for the at least one prior VEGF-targeted therapy was stable disease. In certain instances, the best response for the at least one prior VEGF-targeted therapy was progressive disease. In certain instances, the subject has had at least one prior VEGF-targeted treatment and one or more of: a previous checkpoint inhibitor therapy, a previous interferon therapy, or a previous radiotherapy. In certain embodiments, the subject has had progression of the RCC after the one prior VEGF-targeted treatment. In some instances, the subject has had progression of the RCC within 1, 2, 3, 4, 5, 6, 7, 8, 9, 10, 11, or 12 months of stopping the prior VEGF-targeted treatment. In one embodiment, the subject has had progression of the RCC within 9 months of stopping the prior VEGF-targeted treatment.

The combination therapy involves administering the human subject with RCC with a combination of lenvatinib or a pharmaceutically acceptable salt thereof and everolimus. In certain embodiments, the RCC is an advanced RCC (e.g., advanced renal cell carcinoma following a prior anti-angiogenic therapy). In certain instances, the prior anti-angiogenic therapy is VEGF-targeted therapy. In other embodiments, the RCC is an unresectable advanced RCC. In yet other embodiments, the RCC is a metastatic RCC. In certain embodiments, the RCC in the human subject has one metastasis. In some embodiments, the RCC in the human subject has two metastases. In some embodiments, the RCC in the human subject has three or greater metastases. In certain instances, the site of metastasis/metastases is bone, liver, lung, or lymph nodes. In certain embodiments, the subject belongs to a favorable intermediate Memorial Sloan Kettering Cancer Center (MSKCC) risk group. In certain embodiments, the subject belongs to an intermediate MSKCC risk group. In some embodiments, the subject belongs to a poor MSKCC risk group. In some embodiments, the subject has an Eastern Cooperative Oncology Group (ECOG) performance status of zero. In other embodiments, the subject has an ECOG performance status of one. In certain embodiments, the subject has had a previous nephrectomy.

As shown in Example 1, which describes the results of Phase 2 human clinical trials, the combination of LENVIMA® and everolimus showed a statistically significant and clinically meaningful improvement in progression free survival (PFS) compared with everolimus alone or LENVIMA® alone. In addition, overall survival was longer after treatment with the combination of LENVIMA® and everolimus.

Administration

The combination therapy of everolimus and lenvatinib or a pharmaceutically acceptable salt thereof may be administered to the human subject in need thereof by any means that the health care provider deems useful. For example, each of these compounds may be administered via oral, rectal, nasal, topical (including buccal and sub-lingual), vaginal or parenteral (including intramuscular, sub-cutaneous, and intravenous) administration, or in a form suitable for administration by inhalation, insufflation, or transdermal patch. Each compound may be administered in the same manner or via different methods. Tablets or capsules for oral administration and liquids for intravenous administration are preferred compositions.

For oral administration, the compound can be in the form of, e.g., a tablet, capsule, suspension, or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient(s) may also be administered by injection as a composition wherein, for example, saline, dextrose or water may be used as a suitable pharmaceutically acceptable carrier.

In one embodiment, lenvatinib mesylate is administered to the human subject as a capsule. The capsule can contain, e.g., lenvatinib mesylate equivalent to 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, or 20 mg of lenvatinib. In certain instances, the capsule contains lenvatinib mesylate equivalent to 4 mg lenvatinib. In certain instances, the capsule contains lenvatinib mesylate equivalent to 10 mg lenvatinib. In some embodiments, these capsules also contain one or more of the following inactive ingredients: calcium carbonate, mannitol, microcrystalline cellulose, hydroxypropylcellulose, hydroxypropyl cellulose (type H), and talc. In some embodiments, the shell of these capsules is a hypromellose shell and can contain one or more of: titanium dioxide, ferric oxide yellow, and ferric oxide red. The printing ink used on the capsule may contain one or more of: shellac, black iron oxide, potassium hydroxide, and propylene glycol.

In certain embodiments, lenvatinib mesylate is administered to the human subject at a dose of 18 mg once daily. This dose can be administered, e.g., as one 10 mg capsule and two 4 mg capsules orally once daily. In other embodiments, lenvatinib mesylate is administered to the human subject at a dose of 14 mg once daily. This dose can be administered, e.g., as one 10 mg capsule and one 4 mg capsule orally once daily. In some embodiments, lenvatinib mesylate is administered to the human subject at a dose of 10 mg once daily. This dose can be administered, e.g., as one 10 mg capsule orally once daily. In some embodiments, lenvatinib mesylate is administered to the human subject at a dose of 8 mg once daily. This dose can be administered, e.g., as two 4 mg capsules orally once daily. In some embodiments, lenvatinib mesylate is administered to the human subject at a dose of 6 mg once daily. In some embodiments, lenvatinib mesylate is administered to the human subject at a dose of 4 mg once daily. This dose can be administered, e.g., as one 4 mg capsule orally once daily.

In one embodiment, everolimus is administered to the human subject as a tablet. The tablet can contain, e.g., 1 mg, 2 mg, 2.5 mg, 3 mg, 4 mg, 5 mg, 6 mg, 7 mg, 7.5 mg, 8 mg, 9 mg, or 10 mg of everolimus. In certain instances, the everolimus tablets also contain inactive ingredient(s). For example, the everolimus tablet may contain one or more of: anhydrous lactose, butylated hydroxytoluene, crospovidone, hypromellose, lactose monohydrate, and magnesium stearate as inactive ingredients. In one embodiment, everolimus is administered to the human subject as an oral suspension. The oral suspension may be made by dissolving an everolimus tablet in a liquid (e.g., water). The everolimus tablets for oral suspension also contain inactive ingredient(s). For example, the everolimus tablets for oral suspension can also contain one or more of: butylated hydroxytoluene, colloidal silicon dioxide, crospovidone, hypromellose, lactose monohydrate, magnesium stearate, mannitol, and microcrystalline cellulose as inactive ingredients,

In certain embodiments, everolimus is administered to the human subject at a dose of 5 mg once daily. This dose can be administered, e.g., as one 5 mg tablet orally once daily, two 2.5 mg tablets orally once daily; or an oral suspension of a 5 mg tablet. In some embodiments, everolimus is administered to the human subject at a dose of 5 mg once every other day. In some embodiments, everolimus is administered to the human subject at a dose of 2.5 mg once daily. In some embodiments, everolimus is administered to the human subject at a dose of 2.5 mg once every other day. In certain cases, everolimus is discontinued for Grade 3 toxicity.

In some embodiments, the lenvatinib or a pharmaceutically acceptable salt thereof (e.g., lenvatinib mesylate) is administered as a capsule and everolimus is administered as a tablet. In one embodiment, lenvatinib or pharmaceutically acceptable salt thereof (e.g., lenvatinib mesylate) is administered at a dose of 18 mg once daily and everolimus is co-administered at a dose of 5 mg once daily. In another embodiment, lenvatinib or pharmaceutically acceptable salt thereof is administered at a dose of 18 mg once daily and everolimus is co-administered at a dose of 5 mg once every other day. In one embodiment, lenvatinib or pharmaceutically acceptable salt thereof is administered at a dose of 18 mg once daily and everolimus is co-administered at a dose of 2.5 mg once daily. In another embodiment, lenvatinib or pharmaceutically acceptable salt thereof is administered at a dose of 18 mg once daily and everolimus is co-administered at a dose of 2.5 mg once every other day. In a different embodiment, lenvatinib or pharmaceutically acceptable salt thereof (e.g., lenvatinib mesylate) is administered at a dose of 14 mg once daily and everolimus is co-administered at a dose of 5 mg once daily. In another embodiment, lenvatinib or pharmaceutically acceptable salt thereof is administered at a dose of 14 mg once daily and everolimus is co-administered at a dose of 5 mg once every other day. In a another embodiment, lenvatinib or pharmaceutically acceptable salt thereof is administered at a dose of 14 mg once daily and everolimus is co-administered at a dose of 2.5 mg once daily. In another embodiment, lenvatinib or pharmaceutically acceptable salt thereof is administered at a dose of 14 mg once daily and everolimus is co-administered at a dose of 2.5 mg once every other day. In a different embodiment, lenvatinib or pharmaceutically acceptable salt thereof (e.g., lenvatinib mesylate) is administered at a dose of 10 mg once daily and everolimus is co-administered at a dose of 5 mg once daily. In another embodiment, lenvatinib or pharmaceutically acceptable salt thereof is administered at a dose of 10 mg once daily and everolimus is co-administered at a dose of 5 mg once every other day. In another embodiment, lenvatinib or pharmaceutically acceptable salt thereof is administered at a dose of 10 mg once daily and everolimus is co-administered at a dose of 2.5 mg once daily. In another embodiment, lenvatinib or pharmaceutically acceptable salt thereof is administered at a dose of 10 mg once daily and everolimus is co-administered at a dose of 2.5 mg once every other day. In a different embodiment, lenvatinib or pharmaceutically acceptable salt thereof (e.g., lenvatinib mesylate) is administered at a dose of 8 mg once daily and everolimus is co-administered at a dose of 5 mg once daily. In one embodiment, lenvatinib or pharmaceutically acceptable salt thereof is administered at a dose of 8 mg once daily and everolimus is co-administered at a dose of 5 mg once every other day. In another embodiment, lenvatinib or pharmaceutically acceptable salt thereof is administered at a dose of 8 mg once daily and everolimus is co-administered at a dose of 2.5 mg once daily. In yet another embodiment, lenvatinib or pharmaceutically acceptable salt thereof is administered at a dose of 8 mg once daily and everolimus is co-administered at a dose of 2.5 mg once every other day. In a different embodiment, lenvatinib or pharmaceutically acceptable salt thereof (e.g., lenvatinib mesylate) is administered at a dose of 6 mg once daily and everolimus is co-administered at a dose of 5 mg once daily. In one embodiment, lenvatinib or pharmaceutically acceptable salt thereof is administered at a dose of 6 mg once daily and everolimus is co-administered at a dose of 5 mg once every other day. In another embodiment, lenvatinib or pharmaceutically acceptable salt thereof is administered at a dose of 6 mg once daily and everolimus is co-administered at a dose of 2.5 mg once daily. In yet another embodiment, lenvatinib or pharmaceutically acceptable salt thereof is administered at a dose of 6 mg once daily and everolimus is co-administered at a dose of 2.5 mg once every other day.

It is recommended that the subject take lenvatinib or a pharmaceutically acceptable salt thereof and everolimus one time each day at about the same time, with or without food. In certain embodiments, the subject should not take a CYP3A4 inhibitor and/or a Pgp inhibitor. In certain embodiments, the subject should not take herbal supplements and/or eat grapefruits and/or drink grapefruit juice.

If the patient is unable to swallow the lenvatinib capsules whole, the patient may use a cup to measure about one tablespoon of water or apple juice into a glass and place the drug capsules into the liquid without breaking or crushing them. The capsules should be left in the liquid for at least 10 minutes and the contents then stirred for at least 3 minutes. The patient can then drink this mixture. After drinking, the patient should rinse the glass with a small amount of additional water or apple juice and swallow the liquid.

In certain embodiments, lenvatinib or the pharmaceutically acceptable salt thereof and everolimus are administered to a subject that has a RCC once daily for at least 7 weeks, at least 14 weeks, at least 28 weeks, at least 56 weeks, at least 84 weeks, at least 112 weeks, at least 140 weeks, at least 168 weeks, or at least 196 weeks.

Methods of Treatment to Control, Reduce, or Prevent Adverse Events

A major problem in treating a subject with a new therapy is the development of a treatment-emergent adverse event(s) (TEAE). A treatment-emergent adverse event is as any adverse event not present in the subject prior to the initiation of the treatment, or any adverse event already present that worsens in either intensity or frequency following exposure to the treatment. In certain embodiments, the adverse event is a persistent and intolerable adverse event.

The National Cancer Institute Common Terminology Criteria for Adverse Events v4.0 (CTCAE, published: May 28, 2009; v4.03: Jun. 14, 2010) (incorporated by reference herein in its entirety) is a descriptive terminology that can be utilized for adverse event reporting. The CTCAE provides a grading (severity) scale for each adverse event term. An Adverse Event (AE) is any unfavorable and unintended sign (including an abnormal laboratory finding), symptom, or disease temporally associated with the use of a medical treatment or procedure that may or may not be considered related to the medical treatment or procedure. An AE is a term that is a unique representation of a specific event used for medical documentation and scientific analyses. An AE can be graded. The CTCAE grade refers to the severity of the AE. The CTCAE displays Grades 1 through 5 with unique clinical descriptions of severity for each AE based on this guideline.

Grade 1: Mild; asymptomatic or mild symptoms; clinical or diagnostic observations only; intervention not indicated.

Grade 2: Moderate; minimal, local or noninvasive intervention indicated; limiting age-appropriate instrumental Activities of Daily Living (ADL). [“Instrumental ADL” refer to preparing meals, shopping for groceries or clothes, using the telephone, managing money, etc.]

Grade 3: Severe or medically significant but not immediately life-threatening; hospitalization or prolongation of hospitalization indicated; disabling; limiting self-care ADL. [“Self-care ADL” refers to bathing, dressing and undressing, feeding self, using the toilet, taking medications, and not bedridden.]

Grade 4: Life-threatening consequences; urgent intervention indicated.

Grade 5: Death related to AE.

Not all Grades are appropriate for all AEs. Therefore, some AEs are listed in the CTCAE with fewer than five options for Grade selection.

Combination therapy with lenvatinib or a pharmaceutically acceptable salt thereof can lead to treatment-emergent adverse events (see, Example 2). In certain embodiments, the adverse event associated with therapy with the combination of lenvatinib or a pharmaceutically acceptable salt thereof and everolimus is a persistent and intolerable AE. In certain instances, the persistent and intolerable AE is a Grade 2 AE. In other instances, the persistent and intolerable AE is a Grade 3 AE. In certain embodiments, the adverse event associated with the combination therapy of lenvatinib or a pharmaceutically acceptable salt thereof and everolimus is a Grade 4 AE. In yet other instances, the persistent and intolerable AE is a Grade 4 laboratory abnormality. The most common adverse reactions observed in the LENVIMA®+everolimus-treated subjects were, in order of decreasing frequency, diarrhea, decreased appetite, fatigue, vomiting, nausea, hypertension, cough, and decreased weight.

Diarrhea is a disorder characterized by frequent and watery bowel movements and is graded as follows:

Grade 1: Increase of <4 stools per day over baseline; mild increase in ostomy output compared to baseline.

Grade 2: Increase of 4-6 stools per day over baseline; moderate increase in ostomy output compared to baseline.

Grade 3: Increase of >=7 stools per day over baseline; incontinence; hospitalization indicated; severe increase in ostomy output compared to baseline; limiting self-care ADL.

Grade 4: Life-threatening consequences; urgent intervention indicated.

Grade 5: death.

Fatigue is a disorder characterized by a state of generalized weakness with a pronounced inability to summon sufficient energy to accomplish daily activities and is graded as follows:

Grade 1: Fatigue relieved by rest

Grade 2: Fatigue not relieved by rest; limiting instrumental ADL

Grade 3: Fatigue not relieved by rest, limiting self-care ADL

Grade 4: Not available

Grade 5: Not available

Vomiting is a disorder characterized by the reflexive act of ejecting the contents of the stomach through the mouth, and is graded as follows:

Grade 1: 1-2 episodes (separated by 5 minutes) in 24 hrs

Grade 2: 3-5 episodes (separated by 5 minutes) in 24 hrs

Grade 3: >=6 episodes (separated by 5 minutes) in 24 hrs; tube feeding, TPN or hospitalization indicated

Grade 4: Life-threatening consequences; urgent intervention indicated

Grade 5: Death

Nausea is a disorder characterized by a queasy sensation and/or the urge to vomit, and is graded as follows:

Grade 1: Loss of appetite without alteration in eating habits

Grade 2: Oral intake decreased without significant weight loss, dehydration or malnutrition

Grade 3: Inadequate oral caloric or fluid intake; tube feeding, TPN, or hospitalization indicated

Grade 4: Not available

Grade 5: Not available

Hypertension is a disorder characterized by a pathological increase in blood pressure; a repeatedly elevation in the blood pressure exceeding 140 over 90 mm Hg, and is graded as follows:

Grade 1: Prehypertension (systolic BP 120-139 mm Hg or diastolic BP 80-89 mm Hg)

Grade 2: Stage 1 hypertension (systolic BP 140-159 mm Hg or diastolic BP 90-99 mm Hg); medical intervention indicated; recurrent or persistent (>=24 hrs); symptomatic increase by >20 mm Hg (diastolic) or to >140/90 mm Hg if previously WNL; monotherapy indicated Pediatric: recurrent or persistent (>=24 hrs) BP>ULN; monotherapy indicated

Grade 3: Stage 2 hypertension (systolic BP>=160 mm Hg or diastolic BP>=100 mm Hg); medical intervention indicated; more than one drug or more intensive therapy than previously used indicated Pediatric: Same as adult

Grade 4: Life-threatening consequences (e.g., malignant hypertension, transient or permanent neurologic deficit, hypertensive crisis); urgent intervention indicated Pediatric: Same as adult

Grade 5: Death

Cough is a disorder characterized by sudden, often repetitive, spasmodic contraction of the thoracic cavity, resulting in violent release of air from the lungs and usually accompanied by a distinctive sound, and is graded as follows:

Grade 1: Mild symptoms; nonprescription intervention indicated

Grade 2: Moderate symptoms, medical intervention indicated; limiting instrumental ADL

Grade 3: Severe symptoms; limiting self-care ADL

Grade 4: Not available

Grade 5: Not available

Decreased appetite is a disorder characterized by a loss of appetite, and is graded as follows:

Grade 1: Loss of appetite without alteration in eating habits

Grade 2: Oral intake altered without significant weight loss or malnutrition; oral nutritional supplements indicated

Grade 3: Associated with significant weight loss or malnutrition (e.g., inadequate oral caloric and/or fluid intake); tube feeding or TPN indicated

Grade 4: Life-threatening consequences; urgent intervention indicated

Grade 5: Death

Decreased weight is a finding characterized by a decrease in overall body weight; for pediatrics, less than the baseline growth curve, and is graded as follows:
Grade 1: Weight loss 5 to <10% from baseline; intervention not indicated
Grade 2: 10-<20% from baseline; nutritional support indicated
Grade 3: >=20% from baseline; tube feeding or TPN indicated

Grade 4: Not available

Grade 5: Not available

The most common serious adverse reactions in the LENVIMA®+everolimus-treated group were anemia, dehydration, acute renal failure, diarrhea, and thrombocytopenia.

Anemia is a disorder characterized by a reduction in the amount of hemoglobin in 100 ml of blood. Signs and symptoms of anemia may include pallor of the skin and mucous membranes, shortness of breath, palpitations of the heart, soft systolic murmurs, lethargy, and fatigability, and is graded as follows:

Grade 1: Hemoglobin (Hgb)<LLN—10.0 g/dL; <LLN—6.2 mmol/L; <LLN—100 g/L

Grade 2: Hgb<10.0-8.0 g/dL; <6.2-4.9 mmol/L; <100-80 g/L

Grade 3: Hgb<8.0 g/dL; <4.9 mmol/L; <80 g/L; transfusion indicated

Grade 4: Life-threatening consequences; urgent intervention indicated

Grade 5: Death

Dehydration is a disorder characterized by excessive loss of water from the body. It is usually caused by severe diarrhea, vomiting or diaphoresis, and is graded as follows:

Grade 1: Increased oral fluids indicated; dry mucous membranes; diminished skin turgor

Grade 2: IV fluids indicated <24 hrs

Grade 3: IV fluids or hospitalization indicated

Grade 4: Life-threatening consequences; urgent intervention indicated

Grade 5: Death

Acute renal failure is a disorder characterized by the acute loss of renal function and is traditionally classified as pre-renal (low blood flow into kidney), renal (kidney damage) and postrenal causes (ureteral or bladder outflow obstruction) and is graded as follows:

Grade 1: Creatinine level increase of >0.3 mg/dL; creatinine 1.5-2.0× above baseline

Grade 2: Creatinine 2-3× above baseline

Grade 3: Creatinine>3× baseline or >4.0 mg/dL; hospitalization indicated

Grade 4: Life-threatening consequences; urgent intervention indicated

Grade 5: Death

Thrombocytopenia is a finding based on laboratory test results that indicate a decrease in number of platelets in a blood specimen, and is graded as follows:

Grade 1: <LLN—75,000/mm³; <LLN—75.0×10⁹/L

Grade 2: <75,000-50,000/mm³; <75.0-50.0×10⁹/L

Grade 3: <50,000-25,000/mm³; <50.0-25.0×10⁹/L

Grade 4: <25,000/mm³; <25.0×10⁹/L

Grade 5: Not available

This disclosure provides dose modifications for lenvatinib or a pharmaceutically acceptable salt thereof and/or everolimus upon the occurrence of a treatment-emergent adverse event(s) during the course of the combination therapy. In certain embodiments, the subject that has a RCC is administered a first dosage regimen comprising lenvatinib or a pharmaceutically acceptable salt thereof at a dose of 18 mg/day and everolimus at a dose of 5 mg/day. In other embodiments, the subject that has a RCC is administered a first dosage regimen comprising lenvatinib or a pharmaceutically acceptable salt thereof at a dose of 14 mg/day and everolimus at a dose of 5 mg/day. In some cases, the subject may develop a Grade 1 or tolerable Grade 2 adverse reaction after being administered the first dosage regimen. In such instances, treatment of the subject can continue without any changes to the first dosage regimen. Following or during treatment period with the first dosage regimen comprising lenvatinib or a pharmaceutically acceptable salt thereof at a dose of 14 mg/day and everolimus at a dose of 5 mg/day, if the human subject does not develop an intolerable Grade 2 or Grade 3 adverse reaction or Grade 4 laboratory abnormality, the dosage regimen can be up-titrated (e.g., comprising lenvatinib or a pharmaceutically acceptable salt thereof at a dose of18 mg/day and everolimus at a dose of 5 mg/day).

In some embodiments, the subject may develop a persistent and intolerable Grade 2 or Grade 3 adverse reaction during the period of treatment with the first dosage regimen that is related to one or both compounds of the combination therapy. In certain instances, the subject develops a persistent and intolerable Grade 2 or Grade 3 adverse reaction within 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, or 20 weeks after the administration of the first dosage regimen. In one embodiment, the subject develops a persistent and intolerable Grade 2 or Grade 3 adverse reaction within 12 weeks after the administration of the first dosage regimen. In another embodiment, the subject develops a persistent and intolerable Grade 2 or Grade 3 adverse reaction within 16 weeks after the administration of the first dosage regimen. In certain instances, the AE is diarrhea. In other instances, the AE is vomiting. In yet other instances, the AE is nausea. In still other instances, the AE is proteinuria. In other instances, the AE is decreased appetite. In some instances, the AE is fatigue. In some instances, the AE is hypertension. In some instances, the AE is cough. In other instances, the AE is decreased weight. In certain instances, the Grade 2 or Grade 3 adverse reaction is Grade 3 hypertension, Grade 2 hypertension, Grade 3 cardiac dysfunction, Grade 2 cardiac dysfunction, Grade 3 arterial thromboembolic event, Grade 2 arterial thromboembolic event, Grade 3 proteinuria, Grade 2 proteinuria, Grade 3 renal failure or impairment, Grade 2 renal failure or impairment, Grade 3 diarrhea, Grade 2 diarrhea, Grade 3 gastrointestinal perforation or fistula, Grade 2 gastrointestinal perforation or fistula, Grade 3 vomiting, Grade 2 vomiting, Grade 3 decreased appetite, Grade 2 decreased appetite, Grade 3 fatigue, Grade 2 fatigue, Grade 3 nausea, Grade 2 nausea, Grade 3 cough, Grade 2 cough, Grade 3 decreased weight, Grade 2 decreased weight, Grade 3 dehydration, Grade 2 dehydration, Grade 3 thrombocytopenia, Grade 2 thrombocytopenia, Grade 3 anemia, Grade 2 anemia, Grade 3 acute renal failure, Grade 2 acute renal failure, Grade 3 proteinuria, Grade 2 proteinuria, Grade 3 QT/QTc interval prolongation, Grade 2 QT/QTc interval prolongation, Grade 3 reversible posterior leukoencephalopathy syndrome (RPLS), Grade 2 RPLS, Grade 3 hemorrhagic events, Grade 2 hemorrhagic events, Grade 3 hyperthyroidism, or Grade 2 hyperthyroidism. In some instance, the adverse event may be a Grade 4 laboratory abnormality (e.g., increased lipase, hypertriglyceridemia, hypophosphatemia, high cholesterol, hyponatremia, hypokalemia, hyperkalemia, hypocalcemia, hyperglycemia, increased aspartate aminotransferase, increased alanine aminotransferase, or increased alkaline phosphatase). In certain instances, the Grade 4 laboratory abnormality is Grade 4 increase in aspartate aminotransferase, Grade 4 increase in alanine aminotransferase, Grade 4 increase in alkaline phosphatase, Grade 4 hyperkalemia, Grade 4 hypokalemia, Grade 4 hyponatremia, Grade 4 hypocalcemia, Grade 4 hypophosphatemia, Grade 4 hyperglycemia, Grade 4 hypertriglyceridemia, Grade 4 increase in cholesterol, Grade 4 increase in lipase, Grade 4 decrease in hemoglobin, Grade 4 decrease in platelet count, or Grade 4 decrease in lymphocyte count. In certain instances, the subject develops a Grade 4 AE. In some cases, the Grade 4 AE is renal failure or renal impairment. In some cases, the Grade 4 AE is hepatotoxicity.

If the subject develops a persistent and intolerable Grade 2 or Grade 3 adverse reaction, or a Grade 4 laboratory abnormality after being administered the first dosage regimen (i.e., lenvatinib or a pharmaceutically acceptable salt thereof at a dose of 18 mg/day and everolimus at a dose of 5 mg/day), the healthcare provider can terminate the first dosage regimen and administer to the subject a second dosage regimen comprising lenvatinib or a pharmaceutically acceptable salt thereof at a dose of 14 mg/day. Similarly, if this occurs when the first dosage regimen comprises lenvatinib or a pharmaceutically acceptable salt thereof at a dose of 14 mg/day, the healthcare provider can terminate the first dosage regimen and administer to the subject a second dosage regimen comprising lenvatinib or a pharmaceutically acceptable salt thereof at a dose of 10 mg/day. If the health care provider believes that the adverse reaction to the combination therapy of the first dosage regimen is unrelated to everolimus administration, the second dosage regimen can include everolimus at a dose of 5 mg/day (i.e., lenvatinib or a pharmaceutically acceptable salt thereof at a dose of 14 mg/day and everolimus at a dose of 5 mg/day). If, however, the health care provider believes that the adverse reaction to the lenvatinib+everolimus combination therapy of the first dosage regimen is probably or possibly related to everolimus administration, the healthcare provider can reduce the dosage of everolimus that is administered along with the lenvatinib or a pharmaceutically acceptable salt thereof in the second dosage regimen. For example, if the health care provider believes that the adverse reaction to the lenvatinib+everolimus combination therapy of the first dosage regimen is probably or possibly related to everolimus administration, the second dosage regimen may comprise, e.g., lenvatinib or a pharmaceutically acceptable salt thereof at a dose of 14 mg/day and everolimus at a dose of 5 mg every other day; lenvatinib or a pharmaceutically acceptable salt thereof at a dose of 14 mg/day and everolimus at a dose of 2.5 mg/day; or lenvatinib or a pharmaceutically acceptable salt thereof at a dose of 14 mg/day and everolimus at a dose of 2.5 mg every other day. In certain instances, the second dosage regimen is administered after interruption of the first dosage regimen and after the adverse reaction observed after the first dosage regimen is resolved to Grade 0-1 or baseline (or Grade 0-1 or tolerable Grade 2). In certain instances, the second dosage regimen is administered after interruption of the first dosage regimen and after the adverse reaction observed after the first dosage regimen is resolved to less than or equal to Grade 2. In some instances, the first dosage regimen is terminated only after commencement of medical management of the persistent and intolerable Grade 2 or Grade 3 adverse reaction, or a Grade 4 laboratory abnormality. In specific embodiments, the persistent and intolerable Grade 2 or Grade 3 adverse reaction is diarrhea, vomiting, nausea, or proteinuria. If the persistent and intolerable Grade 2 or Grade 3 adverse reaction is proteinuria, in one embodiment, the therapy with lenvatinib or a pharmaceutically acceptable salt thereof is withheld for ≥2 grams of proteinuria/24 hours, and the treatment with lenvatinib or a pharmaceutically acceptable salt thereof is resumed at a lower dose (e.g., 14 mg/day) when proteinuria is <2 gm/24 hours; however, treatment with lenvatinib or a pharmaceutically acceptable salt thereof is discontinued if the subject develops nephrotic syndrome. If the persistent and intolerable Grade 3 adverse reaction is hypertension, in one embodiment, the subject is provided antihypertensive therapy and treatment with lenvatinib or a pharmaceutically acceptable salt thereof is resumed at a lower dose (e.g., 14 mg/day) when hypertension is controlled at less than or equal to Grade 2; however, therapy with lenvatinib or a pharmaceutically acceptable salt thereof is discontinued for life-threatening hypertension. If the persistent and intolerable Grade 3 adverse reaction is cardiac dysfunction or hemorrhage, in one embodiment, therapy with lenvatinib or a pharmaceutically acceptable salt thereof is withheld until improvement to Grade 0 or 1 or baseline (or Grade 0-1 or tolerable Grade 2) and then the subject is administered lenvatinib or a pharmaceutically acceptable salt thereof at a lower dose (e.g., 14 mg/day); however, therapy with lenvatinib or a pharmaceutically acceptable salt thereof is discontinued if the cardiac dysfunction or hemorrhage is severe and/or persistent. If the adverse reaction is an arterial thrombotic event, therapy with lenvatinib or a pharmaceutically acceptable salt thereof is discontinued. If the subject develops Grade 3 or Grade 4 renal failure/impairment or hepatotoxicity, in one embodiment, treatment with lenvatinib or a pharmaceutically acceptable salt thereof is withheld until the adverse reaction is resolved to Grade 0 to 1 or baseline (or Grade 0-1 or tolerable Grade 2) and resumed at a lower dose (e.g., 14 mg/day); however, therapy with lenvatinib or a pharmaceutically acceptable salt thereof is discontinued if the renal failure/impairment or hepatotoxicity is severe (e.g., hepatic failure) and/or persistent. If the adverse reaction is a gastrointestinal perforation or life-threatening fistula formation, therapy with lenvatinib or a pharmaceutically acceptable salt thereof is discontinued. If the adverse reaction is a Grade 3 or greater QT interval prolongation, therapy with lenvatinib or a pharmaceutically acceptable salt thereof is discontinued and therapy can be resumed at a lower dose (e.g., 14 mg/day) when QT prolongation resolves to Grade 0 or 1 or baseline (or Grade 0-1 or tolerable Grade 2). If the adverse reaction is RPLS, therapy with lenvatinib or a pharmaceutically acceptable salt thereof is discontinued until the adverse reaction is fully resolved; upon resolution, therapy with lenvatinib or a pharmaceutically acceptable salt thereof can be resumed at a lower dose (e.g., 14 mg/day), or discontinued if neurologic symptoms are severe and/or persistent.

In some cases, even after administration of the second dosage regimen, a subject may develop an adverse reaction. In certain instances, the subject develops a persistent and intolerable Grade 2 or Grade 3 adverse reaction within 1 week, 2 weeks, 3 weeks, 4 weeks, 5 weeks, 6 weeks, 7 weeks, 8 weeks, 9 weeks, 10 weeks, 11 weeks, or 12 weeks after the administration of the second dosage regimen. The adverse reaction after the second dosage regimen may be the same as, or different from, the adverse reaction after the first dosage regimen. The adverse reaction after the second dosage regimen may be a persistent and intolerable Grade 2 or Grade 3 adverse reaction. In certain instances, the AE is diarrhea. In other instances, the AE is vomiting. In yet other instances, the AE is nausea. In still other instances, the AE is proteinuria. In other instances, the AE is decreased appetite. In some instances, the AE is fatigue. In some instances, the AE is hypertension. In some instances, the AE is cough. In other instances, the AE is decreased weight. In some embodiments, the adverse event may be a Grade 4 laboratory abnormality (e.g., increased lipase, hypertriglyceridemia, hypophosphatemia, high cholesterol, hyponatremia, hypokalemia, hyperkalemia, hypocalcemia, hyperglycemia, increased aspartate aminotransferase, increased alanine aminotransferase, or increased alkaline phosphatase). In certain instances, the subject develops a Grade 4 AE. In some cases, the Grade 4 AE is renal failure or renal impairment. In some cases, the Grade 4 AE is hepatotoxicity. If the subject develops a persistent and intolerable Grade 2 or Grade 3 adverse reaction, or a Grade 4 laboratory abnormality after being administered the second dosage regimen (e.g., lenvatinib or a pharmaceutically acceptable salt thereof at a dose of 14 mg/day and everolimus at a dose of 5 mg/day), the healthcare provider can terminate the second dosage regimen and administer to the subject a third dosage regimen comprising lenvatinib or a pharmaceutically acceptable salt thereof at a dose of 10 mg/day. Similarly, if this occurs when the second dosage regimen comprises lenvatinib or a pharmaceutically acceptable salt thereof at a dose of 10 mg/day, the healthcare provider can terminate the second dosage regimen and administer to the subject a third dosage regimen comprising lenvatinib or a pharmaceutically acceptable salt thereof at a dose of 8 mg/day. If the health care provider believes that the adverse reaction to the second dosage regimen is unrelated to everolimus administration, the third dosage regimen can include everolimus at a dose of 5 mg/day (i.e., lenvatinib or a pharmaceutically acceptable salt thereof at a dose of 10 mg/day and everolimus at a dose of 5 mg/day). If, however, the health care provider believes that the adverse reaction to the lenvatinib+everolimus combination therapy of the second dosage regimen is probably or possibly related to everolimus administration, the healthcare provider can reduce the dosage of everolimus that is administered along with lenvatinib or a pharmaceutically acceptable salt thereof in the third dosage regimen. For example, if the health care provider believes that the adverse reaction to the second dosage regimen is probably or possibly related to everolimus administration, the third dosage regimen may comprise, e.g., lenvatinib or a pharmaceutically acceptable salt thereof at a dose of 10 mg/day and everolimus at a dose of 5 mg every other day; lenvatinib or a pharmaceutically acceptable salt thereof at a dose of 10 mg/day and everolimus at a dose of 2.5 mg/day; or lenvatinib or a pharmaceutically acceptable salt thereof at a dose of 10 mg/day and everolimus at a dose of 2.5 mg every other day. In certain instances, the third dosage regimen is administered after termination of the second dosage regimen and after the adverse reaction observed after the second dosage regimen is resolved to Grade 0-1 or baseline (or Grade 0-1 or tolerable Grade 2). In some instances, the second dosage regimen is terminated only after commencement of medical management of the persistent and intolerable Grade 2 or Grade 3 adverse reaction, or a Grade 4 laboratory abnormality. In specific embodiments, the persistent and intolerable Grade 2 or Grade 3 adverse reaction is diarrhea, vomiting, nausea, or proteinuria.

In certain embodiments, even after administration of the third dosage regimen, a subject may develop an adverse reaction. In certain instances, the subject develops a persistent and intolerable Grade 2 or Grade 3 adverse reaction within 1 week, 2 weeks, 3 weeks, 4 weeks, 5 weeks, 6 weeks, 7 weeks, 8 weeks, 9 weeks, 10 weeks, 11 weeks, or 12 weeks after the administration of the third dosage regimen. The adverse reaction after the third dosage regimen may be the same as, or different from, the adverse reaction after the second and/or first dosage regimen. The adverse reaction after the third dosage regimen may be a persistent and intolerable Grade 2 or Grade 3 adverse reaction. In certain instances, the AE is diarrhea. In other instances, the AE is vomiting. In yet other instances, the AE is nausea. In still other instances, the AE is proteinuria. In other instances, the AE is decreased appetite. In some instances, the AE is fatigue. In some instances, the AE is hypertension. In some instances, the AE is cough. In other instances, the AE is decreased weight. In some embodiments, the adverse event may be a Grade 4 laboratory abnormality (e.g., increased lipase, hypertriglyceridemia, hypophosphatemia, high cholesterol, hyponatremia, hypokalemia, hyperkalemia, hypocalcemia, hyperglycemia, increased aspartate aminotransferase, increased alanine aminotransferase, or increased alkaline phosphatase). In certain instances, the subject develops a Grade 4 AE. In some cases, the Grade 4 AE is renal failure or renal impairment. In some cases, the Grade 4 AE is hepatotoxicity. If the subject develops a persistent and intolerable Grade 2 or Grade 3 adverse reaction, or a Grade 4 laboratory abnormality, after being administered the third dosage regimen (e.g., lenvatinib or a pharmaceutically acceptable salt thereof at a dose of 10 mg/day and everolimus at a dose of 5 mg/day), the healthcare provider can terminate the third dosage regimen and administer to the subject a fourth dosage regimen comprising lenvatinib or a pharmaceutically acceptable salt thereof at a dose of 8 mg/day. Similarly, if this occurs when the third dosage regimen comprises lenvatinib or a pharmaceutically acceptable salt thereof at a dose of 8 mg/day, the healthcare provider can terminate the third dosage regimen and administer to the subject a fourth dosage regimen comprising lenvatinib or a pharmaceutically acceptable salt thereof at a dose of 4 mg/day. If the health care provider believes that the adverse reaction to the third dosage regimen is unrelated to everolimus administration, the fourth dosage regimen can include everolimus at a dose of 5 mg/day (i.e., lenvatinib or a pharmaceutically acceptable salt thereof at a dose of 8 mg/day and everolimus at a dose of 5 mg/day). If, however, the health care provider believes that the adverse reaction to the lenvatinib+everolimus combination therapy is probably or possibly related to everolimus administration, the healthcare provider can reduce the dosage of everolimus that is administered along with the 8 mg/day of lenvatinib or a pharmaceutically acceptable salt thereof in the fourth dosage regimen. For example, if the health care provider believes that the adverse reaction to the third dosage regimen is probably or possibly related to everolimus administration, the fourth dosage regimen may comprise, e.g., lenvatinib or a pharmaceutically acceptable salt thereof at a dose of 8 mg/day and everolimus at a dose of 5 mg every other day; lenvatinib or a pharmaceutically acceptable salt thereof at a dose of 8 mg/day and everolimus at a dose of 2.5 mg/day; or lenvatinib or a pharmaceutically acceptable salt thereof at a dose of 8 mg/day and everolimus at a dose of 2.5 mg every other day. In certain instances, the fourth dosage regimen is administered after interruption of the third dosage regimen and after the adverse reaction observed after the third dosage regimen is resolved to Grade 0-1 or baseline (or Grade 0-1 or tolerable Grade 2). In some instances, the third dosage regimen is terminated only after commencement of medical management of the persistent and intolerable Grade 2 or Grade 3 adverse reaction, or a Grade 4 laboratory abnormality. In specific embodiments, the persistent and intolerable Grade 2 or Grade 3 adverse reaction is diarrhea, vomiting, nausea, or proteinuria.

In some instances, the subject that has a RCC may have severe renal impairment (creatinine clearance [CLcr] less than 30 mL/min calculated by the Cockcroft-Gault equation) or severe hepatic impairment (Child-Pugh C). For such subjects, the first dose regimen can comprise lenvatinib or a pharmaceutically acceptable salt thereof at a dose of 14 mg/day and everolimus at a dose of 2.5 mg/day. If the subject develops adverse reactions during the course of treatment with the first dose regimen, the administration of the first dosage regimen may be terminated and a lower dosage regimen administered. For example, the subject may be administered a second dosage regimen comprising lenvatinib or a pharmaceutically acceptable salt thereof at a dose of 10 mg/day (and optionally everolimus at a dose of 2.5 mg/day or everolimus at a dose of 2.5 mg every other day). If the subject develops adverse reactions during the course of treatment with the second dose regimen, the administration of the second dosage regimen may be terminated and a lower dosage regimen administered. For example, the subject may be administered a third dosage regimen comprising lenvatinib or a pharmaceutically acceptable salt thereof at a dose of 8 mg/day (and optionally everolimus at a dose of 2.5 mg/day or everolimus at a dose of 2.5 mg every other day). If the subject develops adverse reactions during the course of treatment with the third dose regimen, the administration of the third dosage regimen may be terminated and a lower dosage regimen administered. For example, the subject may be administered a fourth dosage regimen comprising lenvatinib or a pharmaceutically acceptable salt thereof at a dose of 6 mg/day (and optionally everolimus at a dose of 2.5 mg/day or everolimus at a dose of 2.5 mg every other day).

In certain embodiments for treating a subject that has a RCC and also suffers from either severe renal impairment or severe hepatic impairment, the first dose regimen comprises lenvatinib or a pharmaceutically acceptable salt thereof at a dose of 10 mg/day and everolimus at a dose of 2.5 mg/day. If the subject develops adverse reactions during the course of treatment with the first dose regimen, the administration of the first dosage regimen may be terminated and a lower dosage regimen administered. For example, the subject may be administered a second dosage regimen comprising lenvatinib or a pharmaceutically acceptable salt thereof at a dose of 8 mg/day (and optionally everolimus at a dose of 2.5 mg/day or everolimus at a dose of 2.5 mg every other day). If the subject develops adverse reactions during the course of treatment with the second dose regimen, the administration of the second dosage regimen may be terminated and a lower dosage regimen administered. For example, the subject may be administered a third dosage regimen comprising lenvatinib or a pharmaceutically acceptable salt thereof at a dose of 6 mg/day (and optionally everolimus at a dose of 2.5 mg/day or everolimus at a dose of 2.5 mg every other day). If the subject develops adverse reactions during the course of treatment with the third dose regimen, the administration of the third dosage regimen may be terminated and a lower dosage regimen administered. For example, the subject may be administered a fourth dosage regimen comprising lenvatinib or a pharmaceutically acceptable salt thereof at a dose of 5 mg/day (and optionally everolimus at a dose of 2.5 mg/day or everolimus at a dose of 2.5 mg every other day). In certain instance, if Grade 3 toxicity is observed, administration of everolimus can be terminated.

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The following are examples of the practice of the invention. They are not to be construed as limiting the scope of the invention in any way.

### EXAMPLES

### Example 1: Determination of Safety and Efficacy of Lenvatinib Therapies

A multicenter, randomized, open-label, trial was conducted to determine the safety and efficacy of lenvatinib administered alone or in combination with everolimus in subjects with unresectable advanced or metastatic renal cell carcinoma (RCC). The trial consisted of a Phase 1b dose finding and a Phase 2 portion. The Phase 2 portion enrolled a total of 153 patients with unresectable advanced or metastatic RCC following one prior VEGF-targeted treatment. Patients were required, inter alia, to have histological confirmation of predominant clear cell RCC, radiographic evidence of disease progression according to Response Evaluation Criteria in Solid Tumors Version 1.1 (RECIST), one prior VEGF-targeted therapy, and Eastern Cooperative Oncology Group (ECOG) Performance Status of 0 or 1.

Patients were randomly allocated to one of 3 arms: (i) LENVIMA® (mesylate salt of lenvatinib) 18 mg+everolimus 5 mg, (ii) LENVIMA® 24 mg, or (iii) everolimus 10 mg, using a 1:1:1 ratio. Patients were stratified by hemoglobin level (≤13 g/dL vs. >13 g/dL for males and ≤11.5 g/dL vs >11.5 g/dL for females) and corrected serum calcium (≥10 mg/dL vs. <10 mg/dL).

The primary efficacy outcome measure, based on investigator assessed tumor response, was progression-free survival (PFS) of the LENVIMA® plus everolimus arm vs. the everolimus arm and of the LENVIMA® arm vs. the everolimus arm. Other efficacy outcome measures included investigator-assessed overall survival (OS) and objective response rate (ORR).

Of the 153 patients randomly allocated, 73% were male, the median age was 61 years, 36% were older than 65 years, and 97% were white. Metastases were present in 95% of the patients and unresectable advanced disease was present in 5%. All patients had a baseline Eastern Cooperative Oncology Group (ECOG) performance status (PS) of either 0 (55%) or 1 (45%) with similar distribution across the 3 treatment arms. Memorial Sloan Kettering Cancer Center (MSKCC) poor risk was observed in 39% of patients in the LENVIMA®+everolimus arm, 44% in the LENVIMA® arm, and 38% in the everolimus arm. The median time from diagnosis to first dose was 32 months in the LENVIMA®+everolimus-treatment arm, 33 months in the LENVIMA® arm, and 26 months in the everolimus arm.

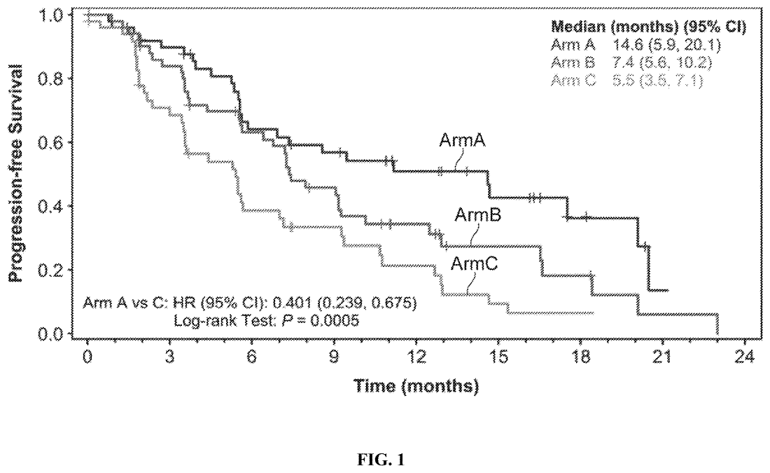

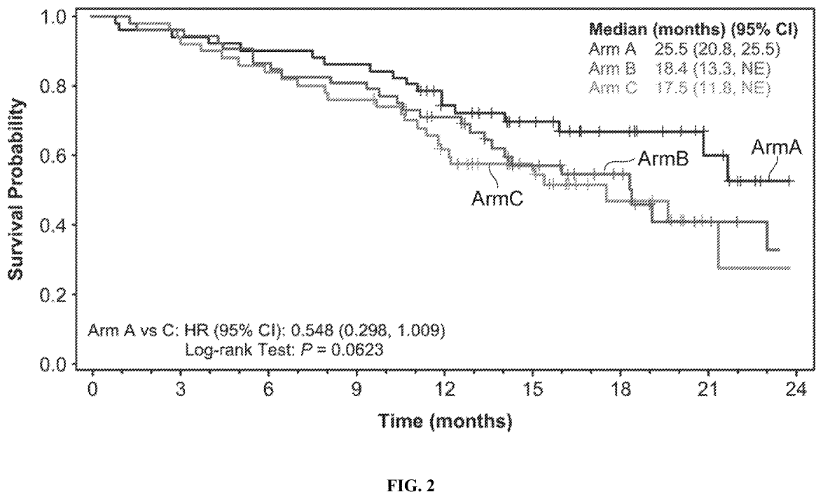

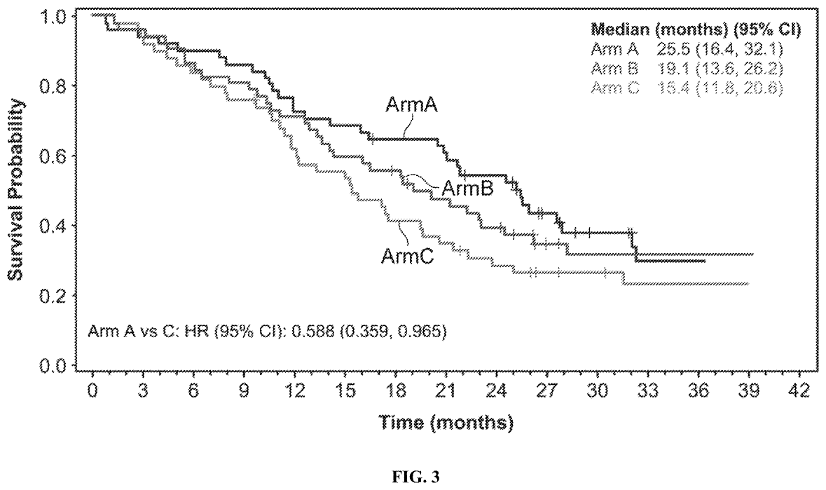

Tumor assessments were evaluated according to RECIST. The LENVIMA®+everolimus arm showed a statistically significant and clinically meaningful improvement in PFS compared with the everolimus arm (see, Table 1 and FIG. 1). The LENVIMA arm also showed an improvement in PFS compared with the everolimus arm (see, Table 1 and FIG. 1). Overall survival was longer in the LENVIMA®+everolimus arm; a trend that was maintained through the cutoff for the updated OS analysis (see, Table 1 and FIGS. 2 and 3).

TABLE 1
Efficacy Results in Renal Cell Carcinoma (Investigator Assessment)

| | | |
|---|---|---|
| LENVIMA ® 18 mg + | | |
| Everolimus 5 mg | LENVIMA ® 24 mg | Everolimus 10 mg |
| (N = 51) | (N = 52) | (N = 50) |

Progression-FreeSurvival (PFS)^{a}

| | | | |
|---|---|---|---|
| Median PFS in months (95% CI) | 14.6 (5.9, 20.1) | 7.4 (5.6, 10.2) | 5.5 (3.5, 7.1) |
| Hazard Ratio (95% CI)^{b} | 0.40 (0.24, 0.68) | | |
| LENVIMA ® + Everolimus vs | | | |
| Everolimus | | | |
| P Value | 0.0005 | — | — |
| LENVIMA ® + Everolimus vs | | | |
| Everolimus | | | |

Overall Survival (OS)

| | | | |
|---|---|---|---|
| Number of deaths (%) | 19 (37) | 26 (50) | 26 (52) |
| Median OS in months (95% CI) | 25.5 (20.8, 25.5) | 18.4 (13.3, NE) | 17.5 (11.8, NE) |
| Hazard Ratio (95% CI)^{b} | 0.55 (0.30, 1.01) | — | — |
| LENVIMA ® + Everolimus vs | | | |
| Everoliruus | | | |
| P Value^{b} | 0.0623 | — | — |
| LENVIMA ® + Everolimus vs | | | |
| Everolimus | | | |

Overall Survival (updated analysis)^{c}

| | | | |
|---|---|---|---|
| Number of deaths, n (%) | 32 (63) | 34 (65) | 37 (74) |
| Median OS in months (95% CI) | 25.5 (16 .4, 32.1) | 19.1 (13.6, 26.2) | 15.4 (11.8, 20.6) |
| Hazard Ratio (95% CI)^{b} | 0.59 (0.36, 0.97) | | |
| LENVIMA ® + Everolimus vs | | | |
| Everolimus | | | |

Objective Response Rate

| | | | |
|---|---|---|---|
| Number of complete responses (%) | 1 (2) | 0 | 0 |
| Number of partial responses (%) | 21 (41) | 14 (27) | 3 (6) |
| Number of stable disease (%) | 21 (41) | 27 (52) | 31 (62) |
| Number of progressive disease (%) | 2 (4) | 3 (6) | 12 (24) |
| Duration of response, months, | 13.0 (3.7, NE) | 7.5 (3.8, NE) | 8.5 (7.5, 9.4) |
| median (95% CI) | | | |

Tumor assessment was based on RECIST 1.1 criteria. Data cutoff date = Jun. 13, 2014
Percentages are based on the total number of subjects in the Full Analysis Set within relevant treatment group.
CI = confidence interval, NE = not estimable.
^{a}Point estimates are based on Kaplan-Meier method and 95% CIs are based on the Greenwood formula using log-log transformation.
^{b}Stratified hazard ratio is based on a stratified Cox regression model including treatment as a covariate factor and hemoglobin and corrected serum calcium as strata. The Effron method was used for correction for tied events.
^{c}Data cutoff date = Jul. 31, 2015

The treatment effect of LENVIMA®+everolimus on PFS and Overall Response Rate (ORR) was also supported by a retrospective independent blinded review of scans. The LENVIMA®+everolimus arm showed a statistically significant and clinically meaningful improvement in PFS (Hazard ratio [HR]=0.50, [95% CI: 0.26, 0.79], P=0.003) compared with the everolimus arm. Results for ORR were consistent with that of the investigators' assessments, 35.3% in the LENVIMA®+everolimus arm, with 1 complete response and 17 partial responses; no subject had an objective response in the everolimus arm (P value<0.0001) in favor of the LENVIMA®+everolimus arm.

### Example 2: Adverse Reactions in Trial Examining Lenvatinib Therapies for RCC

The safety data described below are derived from the trial described in Example 1, which randomized (1:1:1) patients with unresectable advanced or metastatic renal cell carcinoma (RCC) to LENVIMA® 18 mg+everolimus 5 mg (n=51), LENVIMA® 24 mg (n=52), or everolimus 10 mg (n=50) once daily. The median treatment duration was 7.6 months for LENVIMA®+everolimus, 7.4 months for LENVIMA® and 4.1 months for everolimus. Among 51 patients who received LENVIMA®+everolimus in this trial, the median age was 61 years, 69% were men, and 98% were white.

In this trial, the most common adverse reactions observed in the LENVIMA®+everolimus-treated group (greater than or equal to 30%) were, in order of decreasing frequency, diarrhea, decreased appetite, fatigue, vomiting, nausea, hypertension, cough, and decreased weight. The most common serious adverse reactions (at least 5%) were anemia 4 (8%), dehydration 4 (8%), acute renal failure 3 (6%), diarrhea 3 (6%), and thrombocytopenia 3 (6%).

Adverse reactions led to dose reductions or interruption in 88% of patients receiving LENVIMA®+everolimus, 79% in patients receiving LENVIMA®, and 54% in patients receiving everolimus. Of the 44 LENVIMA®+everolimus combination therapy patients who dose was reduced or interrupted because of an adverse event, most of them (31) did not have to discontinue entirely for that adverse event. 13 (26%) patients discontinued treatment in the LENVIMA®+everolimus-treated group, 16 (31%) patients discontinued treatment in the LENVIMA®-treated group, and 6 (12%) patients discontinued treatment in the everolimus-treated group for adverse reactions. The most common adverse reactions (at least 5%) resulting in dose reductions in the LENVIMA®+everolimus-treated group were diarrhea 12 (24%), vomiting 4 (8%), nausea 3 (6%), and proteinuria 3 (6%). Table 2 and Table 3 presents the percentage of patients in the trial experiencing adverse reactions with a frequency of at least 10%.

TABLE 2
Adverse Reactions with Frequency of Greater than 10%
for All-Grades or Greater than 3% for Grade 3 and 4

| | LENVIMA ® 18 mg + | | | | | |
|---|---|---|---|---|---|---|
| | Everolimus 5 mg | | LENVIMA ® 24 mg | | Everolimus 10 mg | |
| | (N = 51) | | (N = 52) | | (N = 50) | |
| System Organ Class | All | Grades | All | Grades | All | Grades |
| Preferred Term | Grades (%) | 3-4 (%) | Grades (%) | 3-4 (%) | Grades (%) | 3-4 (%) |

Gastrointestinal Disorders

| | | | | | | |
|---|---|---|---|---|---|---|
| Constipation | 12 | 0 | 37 | 0 | 18 | 0 |
| Diarrhea | 84 | 20 | 71 | 12 | 34 | 2 |
| Dyspepsia | 12 | 0 | 12 | 2 | 12 | 0 |
| Gastrointestinal disorders and | 39 | 4 | 33 | 4 | 8 | 0 |
| Abdominal pain^{a} | | | | | | |
| Nausea | 43 | 6 | 62 | 8 | 16 | 0 |
| Oral inflammation^{b} | 39 | 0 | 27 | 2 | 50 | 4 |
| Oral pain^{c} | 18 | 0 | 14 | 0 | 4 | 0 |
| Vomiting | 47 | 8 | 39 | 4 | 12 | 0 |

General Disorders and Administration Site Conditions

| | | | | | | |
|---|---|---|---|---|---|---|
| Fatigue^{d} | 63 | 16 | 52 | 10 | 38 | 2 |
| Peripheral edema | 29 | 0 | 17 | 0 | 18 | 0 |
| Pyrexia | 22 | 2 | 10 | 0 | 10 | 2 |

Infections and Infestations

| | | | | | | |
|---|---|---|---|---|---|---|
| Dental and oral infections^{e} | 14 | 0 | 6 | 2 | 0 | 0 |

Metabolism and Nutrition Disorders

| | | | | | | |
|---|---|---|---|---|---|---|
| Decreased appetite | 53 | 6 | 58 | 4 | 18 | 0 |
| Decreased weight | 31 | 2 | 50 | 6 | 8 | 0 |
| Dehydration | 10 | 6 | 2 | 0 | 2 | 0 |

Musculoskeletal and Connective Tissue Disorders

| | | | | | | |
|---|---|---|---|---|---|---|
| Arthralgia/myalgia^{f} | 53 | 4 | 56 | 4 | 32 | 0 |
| Musculoskeletal chest pain | 18 | 2 | 15 | 4 | 4 | 0 |

Nervous System Disorders

| | | | | | | |
|---|---|---|---|---|---|---|
| Headache | 18 | 2 | 27 | 6 | 10 | 2 |

Psychiatric Disorders

| | | | | | | |
|---|---|---|---|---|---|---|
| Confusional state | 4 | 4 | 4 | 2 | 0 | 0 |
| Insomnia | 18 | 2 | 15 | 0 | 2 | 0 |

Renal and Urinary Disorders

| | | | | | | |
|---|---|---|---|---|---|---|
| Proteinuria | 24 | 4 | 31 | 19 | 14 | 2 |
| Renal failure events^{g} | 6 | 6 | 10 | 6 | 2 | 2 |

TABLE 3
Additional Adverse Reactions with Frequency of Greater than
10% for All-Grades or Greater than 3% for Grade 3 and 4

| | LENVIMA ® 18 mg + | | | | | |
|---|---|---|---|---|---|---|
| | Everolimus 5 mg | | LENVIMA ® 24 mg | | Everolimus 10 mg | |
| | (N = 51) | | (N = 52) | | (N = 50) | |
| System Organ Class | All | Grades | All | Grades | All | Grades |
| Preferred Term | Grades (%) | 3-4 (%) | Grades (%) | 3-4 (%) | Grades (%) | 3-4 (%) |

Respiratory, Thoracic and Mediastinal Disorders

| | | | | | | |
|---|---|---|---|---|---|---|
| Cough | 39 | 0 | 17 | 2 | 30 | 0 |
| Dysphonia | 20 | 0 | 37 | 0 | 4 | 0 |
| Dyspnea^{h} | 29 | 2 | 25 | 2 | 28 | 8 |
| Epistaxis | 18 | 0 | 8 | 0 | 24 | 0 |

Skin and subcutaneous Tissue Disorders

| | | | | | | |
|---|---|---|---|---|---|---|
| Pruritus | 14 | 0 | 6 | 0 | 14 | 0 |
| Rashⁱ | 22 | 0 | 15 | 0 | 30 | 0 |

Vascular Disorders

| | | | | | | |
|---|---|---|---|---|---|---|
| Hypertension^{j} | 43 | 14 | 48 | 17 | 10 | 2 |

^{a}Includes abdominal discomfort, abdominal pain, lower abdominal pain, upper abdominal pain, abdominal tenderness, epigastric discomfort, and gastrointestinal pain
^{b}Includes aphthous stomatitis, stomatitis, glossitis, mouth ulceration, and mucosal inflammation
^{c}Includes oral pain, glossodynia, and oropharyngeal pain
^{d}Includes asthenia, fatigue, and malaise
^{e}Includes gingivitis, oral infection, parotitis, percoronitis, periodontitis, sialoadenitis, tooth abscess and tooth infection
^{f}Includes musculoskeletal pain, back pain, pain in extremity, arthralgia, and myalgia
^{g}Includes prerenal failure, renal failure, acute renal failure, and renal tubular necrosis
^{h}Includes dyspnea, exertional dyspnea
ⁱIncludes macular rash, maculo-papular rash, generalized rash, and rash
^{j}Includes hypertension, hypertensive crisis, increased diastolic blood pressure and increased blood pressure

Some of the adverse events noted above are addressed below with suggestions for dealing with them.

Hypertension

In the trial described in Example 1, hypertension was reported in 43% of patients in the LENVIMA®+everolimus-treated group (the incidence of Grade 3 or Grade 4 hypertension was 14%), 48% of patients in the LENVIMA®-treated group (the incidence of Grade 3 or Grade 4 hypertension was 17%), and 10% of patients in the everolimus-treated group (the incidence of Grade 3 or Grade 4 hypertension was 2%).

In view of these findings, it would be beneficial to control blood pressure prior to treatment of patients with LENVIMA®+everolimus. The blood pressure of the patient should generally be monitored after 1 week, then every 2 weeks for the first 2 months, and then at least monthly thereafter during treatment with LENVIMA®+everolimus. It would be advisable to withhold LENVIMA® for Grade 3 hypertension despite optimal antihypertensive therapy; and to resume at a reduced dose when hypertension is controlled at less than or equal to Grade 2. Furthermore, LENVIMA® should be discontinued for life-threatening hypertension. Everolimus dose interruption or alternate day dosing should also be considered until toxicity resolves.

Patients undergoing treatment with LENVIMA®+everolimus should be advised by the health care provider to undergo regular blood pressure monitoring and to contact their health care provider if their blood pressure is elevated.

Cardiac Dysfunction

In the trial described in Example 1, decreased ejection fraction and cardiac failure was reported in 4% (2% Grade 3) of patients in the LENVIMA®+everolimus-treated group, 8% of patients in the LENVIMA®-treated group (2% Grade 3), and 4% of patients in the everolimus-treated group (2% Grade 3).

In view of these findings, it would be beneficial to monitor patients for clinical symptoms or signs of cardiac decompensation. It would also be advisable to withhold LENVIMA® for development of Grade 3 cardiac dysfunction until improved to Grade 0 or 1 or baseline. One could then either resume at a reduced dose or discontinue LENVIMA® depending on the severity and persistence of cardiac dysfunction. For Grade 4 cardiac dysfunction, LENVIMA® should be discontinued. Everolimus dose interruption or alternate day dosing should also be considered until toxicity resolves.

Patients undergoing treatment with LENVIMA®+everolimus should be advised by the health care provider that LENVIMA® can cause cardiac dysfunction and to immediately contact their healthcare provider if they experience any clinical symptoms of cardiac dysfunction such as shortness of breath or swelling of ankles.

Arterial Thromboembolic Events

In the above-described trial, no arterial thromboembolic events were observed in the LENVIMA®+everolimus-treated group. 8% of patients in the LENVIMA®-treated group (8% Grade 3 or greater) and 6% of patients in the everolimus-treated group (4% Grade 3 or greater) had arterial thromboembolic events reported.

LENVIMA® should be discontinued following an arterial thrombotic event.

Patients undergoing treatment with LENVIMA®+everolimus should be advised by the health care provider to seek immediate medical attention for new onset chest pain or acute neurologic symptoms consistent with myocardial infarction or stroke.

Hepatotoxicity

In the RCC trial described above, 4% of patients in the LENVIMA®+everolimus-treated group, 4% of patients in the LENVIMA®-treated group, and 2% of patients in the everolimus-treated group experienced an increase in alanine aminotransferase (ALT) that was Grade 3 or greater. 4% of patients in the LENVIMA®+everolimus-treated group, 4% of patients in the LENVIMA®-treated group and no patients in the everolimus-treated group experienced an increase in aspartate aminotransferase (AST) that was Grade 3 or greater.

It is recommended that liver function be monitored before initiation of LENVIMA®, then every 2 weeks for the first 2 months, and at least monthly thereafter during treatment. LENVIMA® should be withheld for the development of Grade 3 or greater liver impairment until resolved to Grade 0 to 1 or baseline. One can then either resume at a reduced dose or discontinue LENVIMA® depending on the severity and persistence of hepatotoxicity. LENVIMA® should be discontinued for hepatic failure.

Patients undergoing treatment with LENVIMA®+everolimus should be advised by the health care provider to undergo lab tests to monitor for liver function and to report any new symptoms indicating hepatic toxicity or failure.

Proteinuria

In this RCC trial, proteinuria was reported in 24% of patients in the LENVIMA®+everolimus-treated group (4% were Grade 3 or greater), 31% of patients in the LENVIMA®-treated group (19% were Grade 3 or greater), and 14% of patients in the everolimus-treated group (2% were Grade 3 or greater).

It is suggested that the healthcare provider monitor for proteinuria before initiation of, and periodically throughout treatment. If urine dipstick proteinuria greater than or equal to 2+ is detected, the health care provider should obtain a 24 hour urine protein. LENVIMA® should be withheld for ≥2 grams of proteinuria/24 hours and treatment with LENVIMA® may be resumed at a reduced dose when proteinuria is <2 gm/24 hours. LENVIMA® should be discontinued for nephrotic syndrome.

Patients undergoing treatment with LENVIMA®+everolimus should be advised by the health care provider to undergo regular lab tests to monitor for kidney function and protein in the urine.

Renal Failure and Impairment/Diarrhea

Events of renal impairment were reported in 18% of LENVIMA®+everolimus-treated group, 15% in the LENVIMA®-treated group, and 12% in the everolimus-treated group. The incidence of Grade 3 or greater renal failure or impairment was 8% in the LENVIMA®+everolimus-treated group, 6% in the LENVIMA®-treated group, and 2% in the everolimus-treated group. Grade 3 or greater diarrhea was reported in 22% of patients in the LENVIMA®+everolimus-treated group, 12% of patients in the LENVIMA®-treated group and 2% of patients in the everolimus-treated group.

The primary risk factor for severe renal impairment in LENVIMA®-treated patients was dehydration/hypovolemia due to diarrhea and vomiting. Active management of diarrhea and any other gastrointestinal symptoms should be initiated for Grade 1 events.

It is recommended that the health care provider withhold LENVIMA® upon development of Grade 3 or 4 renal failure/impairment until it has resolved to Grade 0 to 1 or baseline. One can then either resume at a reduced dose or discontinue LENVIMA® depending on the severity and persistence of renal impairment.

Patients undergoing treatment with LENVIMA®+everolimus should be advised by the health care provider to undergo regular lab tests to monitor for kidney function and protein in the urine.

Gastrointestinal Perforation and Fistula Formation

In the RCC trial, events of gastrointestinal perforation or fistula were reported in 2% of patients in the LENVIMA®+everolimus-treated group, 6% in the LENVIMA®-treated group, and no patients in the everolimus-treated group.

It is recommended that the health care provider discontinue LENVIMA® in patients who develop gastrointestinal perforation or life-threatening fistula.

Patients undergoing treatment with LENVIMA®+everolimus should be advised by the health care provider that LENVIMA® can increase the risk of gastrointestinal perforation or fistula and to seek immediate medical attention for severe abdominal pain.

QT Interval Prolongation

In the above-described trial, QT/QTc interval prolongation was reported in 6% of patients in both the LENVIMA®+everolimus-treated group and the LENVIMA®-treated group. No Grade 3 or greater events were reported in either group. No reports of QT/QTc interval prolongation occurred in the everolimus-treated group.

In view of the above findings, it is recommended that the healthcare provider monitor electrocardiograms in patients with congenital long QT syndrome, congestive heart failure, bradyarrhythmias, or those who are taking drugs known to prolong the QT interval, including Class Ia and III antiarrhythmics. In addition, it is recommended that the healthcare provider monitor and correct electrolyte abnormalities in all patients. LENVIMA® should be withheld upon the development of Grade 3 or greater QT interval prolongation. LENVIMA® administration may be resumed at a reduced dose when QT prolongation resolves to Grade 0 or 1 or baseline.

Hypocalcemia

In the RCC trial, 6% of patients in the LENVIMA®+everolimus-treated group, no patients in the LENVIMA®-treated group, and 2% of patients in the everolimus-treated group experienced Grade 3 or greater hypocalcemia.

Thus, it is recommended that patients be monitored for blood calcium levels at least monthly and that calcium be replaced as necessary during LENVIMA® treatment. LENVIMA® dosing can be interrupted and adjusted as necessary depending on severity, presence of ECG changes, and persistence of hypocalcemia.

Reversible Posterior Leukoencephalopathy Syndrome

In the RCC trial, one patient out of 103 who received either LENVIMA®+everolimus or LENVIMA® monotherapy experienced reversible posterior leukoencephalopathy syndrome (RPLS).

It is prudent to confirm a diagnosis of RPLS with MRI and to withhold for a diagnosis of RPLS treatment with LENVIMA® until the RPLS is fully resolved. Upon resolution, administration of LENVIMA® may resume at a reduced dose or discontinued depending on the severity and persistence of neurologic symptoms.

Hemorrhagic Events

In the RCC trial, hemorrhagic events occurred in 33% of patients in the LENVIMA®+everolimus-treated group (8% were Grade 3 or greater), with the most frequently reported hemorrhagic event being epistaxis (18% Grade 1 only). Discontinuation of treatment due to hemorrhagic events occurred in 4% of patients in the LENVIMA®+everolimus-treated group. In the LENVIMA®-treated group, hemorrhagic events occurred in 29% of patients (2% were Grade 3 or greater) with the most frequently reported hemorrhagic event being epistaxis (8% Grade 1 only). Discontinuation due to hemorrhagic events occurred in 2% of patients in the LENVIMA®-treated group. There was one case of fatal cerebral hemorrhage in the LENVIMA®+everolimus-treated group and one case of fatal intracranial hemorrhage in the LENVIMA®-treated group. In the everolimus-treated group, hemorrhagic events occurred in 28% of patients (2% were Grade 3 or greater) with the most frequently reported hemorrhagic event being epistaxis (20% Grade 1, and 4% Grade 2 only). There were no discontinuations due to hemorrhagic events in the everolimus-treated group.

The healthcare provider should consider withholding LENVIMA® for the development of Grade 3 hemorrhage until resolved to Grade 0 to 1. Treatment with LENVIMA® may be resumed at a reduced dose or discontinued depending on the severity and persistence of hemorrhage. LENVIMA® treatment should be discontinued in patients who experience Grade 4 hemorrhage.

Patients undergoing treatment with LENVIMA®+everolimus should be advised by the health care provider that LENVIMA® can increase the risk for bleeding and to contact their health care provider for bleeding or symptoms of severe bleeding.

Impairment of Thyroid Stimulating Hormone Suppression/Thyroid Dysfunction

In the RCC trial, hypothyroidism occurred in 24% of patients in the LENVIMA®+everolimus-treated group, 37% of patients in the LENVIMA®-treated group, and 2% of patients in the everolimus-treated group. All events of hypothyroidism in the LENVIMA®+everolimus-treated group were of Grade 1 or 2.

75% of patients in the LENVIMA®+everolimus-treated group were not receiving exogenous thyroid replacement, and of these, 71% had normal baseline TSH levels. In patients with a normal TSH at baseline, an elevation of TSH level was observed post baseline in 63% of LENVIMA®+everolimus-treated patients as compared with none in patients receiving everolimus alone.

Thyroid function should be monitored before initiation of, and periodically throughout, treatment with LENVIMA®. Hypothyroidism should be treated according to standard medical practice to maintain euthyroid state.

Embryofetal Toxicity

Based on its mechanism of action and data from animal reproduction studies, LENVIMA® can cause fetal harm when administered to a pregnant woman. In animal reproduction studies, oral administration of lenvatinib during organogenesis at doses below the recommended human dose resulted in embryotoxicity, fetotoxicity, and teratogenicity in rats and rabbits.

Accordingly, the healthcare provider must advise pregnant women of the potential risk to a fetus. In addition, in the case of females of reproductive potential, the healthcare provider should advise the patient to use effective contraception during treatment with LENVIMA® and for at least 2 weeks following completion of therapy.

Patients undergoing treatment with LENVIMA®+everolimus should be advised by the health care provider to inform their healthcare provider of a known or suspected pregnancy.

Table 4 provides a summary of laboratory abnormalities observed in the patients in the RCC trial described in Example 1.

TABLE 4
Laboratory Abnormalities with at Least Greater than or equal to 3% in Grade 3
or 4 Events in LENVIMA ® + Everolimus-Treated Patients^{ab}

| | LENVIMA ® 10 mg + | | Everolimus |
|---|---|---|---|
| | Everolimus 5 mg | LENVIMA ® 24 mg | 10 mg |
| | (N = 51) | (N = 52) | (N = 50) |
| | Grades | Grades | Grades |
| Laboratory Abnormality | 3-4 (%) | 3-4 (%) | 3-4 (%) |

Chemistry

| | | | |
|---|---|---|---|
| Aspartate aminotransferase (AST) | 4 | 4 | 0 |
| increased | | | |
| Alanine aminotransferase (ALT) | 4 | 4 | 2 |
| increased | | | |
| Alkaline phosphatase increased | 4 | 0 | 0 |
| Hyperkalemia | 6 | 2 | 2 |
| Hypokalemia | 8 | 8 | 2 |
| Hyponatremia | 10 | 6 | 6 |
| Hypocalcemia | 6 | 0 | 2 |
| Hypophosphatemia | 14 | 4 | 6 |
| Hyperglycemia | 4 | 8 | 16 |
| Hypertriglyceridemia | 14 | 10 | 18 |
| High cholesterol | 12 | 4 | 0 |
| Lipase increased | 16 | 10 | 12 |

Hematology

| | | | |
|---|---|---|---|
| Hemoglobin decreased | 8 | 4 | 16 |
| Platelet count decreased | 6 | 2 | 0 |
| Lymphocyte count decreased | 6 | 4 | 20 |

^{a}With at least 1 grade increase from baseline
^{b}Subject with at least 1 post baseline laboratory value

### Example 3: Determination of the Safety and Efficacy of Dosing Regimens of Lenvatinib at Two Different Starting Doses (18 mg Vs 14 mg QD) in Combination with Everolimus (5 mg QD) in Renal Cell Carcinoma

This study was a randomized, open-label (formerly double-blind), Phase 2 trial to assess safety and efficacy of Lenvatinib at two different starting doses (18 mg vs 14 mg QD) in combination with Everolimus (5 mg QD) in RCC following one prior VEGF-targeted treatment.

The study included 343 patients (N=343) with the following characteristics:
Predominant clear cell RCC
One prior disease progression to VEGF-targeted treatment
Prior PD-1/PD-L1 treatment is allowed.
Measurable disease at baseline.
KPS≥70.

A total of 343 patients over the study period (which lasted about 48 months) were split into two approximately equal cohorts. Each received continuous daily dosing, with dose reductions for toxicity, until the last enrolled patient reached 6 months (week 24).

One cohort received Lenvatinib® 18 mg QD and Everolimus 5 mg QD. The second cohort received Lenvatinib 14 mg QD and Everolimus 5 mg QD. In the second cohort on C2 Day 1, Lenvatinib® was escalated to 18 mg QD if no intolerable Gr2 or ≥Gr3 AEs that require does reduction were observed in the first 4 weeks.

Stratification factors included:
MSKCC prognostic groups (favourable, intermediate, and poor risk)
Prior PD-1/PD-L1 treatment (yes, no)

The primary efficacy endpoint of this study was the Overall Response Rate after 24 weeks of treatment (ORR_{24W}).

The primary safety endpoint of this study was the proportion of subjects with intolerable Grade 2 or any ≥Grade 3 TEAEs within 24 weeks of treatment.

The secondary endpoints of this study were the progression free survival (PFS), ORR, overall survival (OS), time to the second objective disease progression (PFS2), the proportion of subjects who discontinue the treatment due to AE, the time to treatment failure due to AE and safety.

The exploratory endpoints of the study were ORR_{24W}, ORR, and PFS based on investigator-initiated review (IIR) assessments.

Study Background

In May 2018, Eisai's Clinical Trials Supplies department detected that some subjects, whose lenvatinib dose had previously been reduced, had been reassigned in error to a higher dose level by the IxRS. It affected a total of 32 subjects: 30 subjects were incorrectly up titrated (lenvatinib dose increased by 1-3 dose levels per protocol), and 2 subjects who should have been dose reduced per investigator decision, were instead dispensed the same dose. None of the assigned dose levels exceeded the maximum dose per protocol of 18 mg daily. Initial assessment identified the issue was caused by a post-production Oracle IxRS database change in March 2018. In July 2018, one more subject received a single incorrect lenvatinib dose due to IxRS issues. This led to the decision to unblind the study to study subjects and investigator site personnel which was agreed with the FDA and EMA. Approximately 306 subjects were originally planned to be randomized into the study. Since there were 32 subjects who received ≥2 incorrect lenvatinib doses due to IxRS issues, the number of subjects to be randomized was increased by 32 to a total of approximately 338. Therefore, there were approximately 306 subjects in the Per-Protocol Analysis Set 1.

Analysis Sets for Efficacy and Safety

Efficacy

Per-Protocol Analysis Set 1 (primary population for efficacy): 32 subjects excluded due to receiving incorrect lenvatinib doses due to IxRS issues.

Full Analysis Set (secondary population for efficacy): All randomized subjects, this will be a secondary analysis set for efficacy endpoints.

Per-Protocol Analysis Set 2 (secondary population for efficacy): all subjects who received at least 1 dose of study drug, had no important protocol deviations, and had both baseline and at least 1 postbaseline tumor assessment.

Safety

Safety Analysis Set: all subjects who were randomized and received at least 1 dose of study drug.

Per-Protocol Safety Analysis Set (primary population for primary safety endpoint): all treated subjects in PPAS1.

Per-Protocol Analysis Set 2 (secondary population for primary safety endpoint).

Primary Analysis for Efficacy and Safety Endpoints

Primary Efficacy (ORR₂₄, investigator, confirmed) by:

A non-inferiority test, wherein Ho is odds ratio of ORR_{24W-14mg} VS ORR_{24W-18mg}≤0.76 and Ha is Odds ratio of ORR_{24W-14mg} VS ORR_{24W-18mg}>0.76 (non-inferiority). Odds Ratio: Cochran-Mantel-Haenszel (CMH) test stratified by the Memorial Sloan-Kettering Cancer Center clinical risk score (MSKCC) and prior PD-1/PD-L1 treatment from IxRS. Alpha level: If 1-sided P value is ≤0.045, non-inferiority in ORR_{24W} will be claimed.

Primary Safety:

Proportion of subjects with intolerable Grade 2 or any ≥Grade 3 TEAEs within 24 weeks. If non-inferiority in ORR_{24W} is claimed, the superiority test at 2-sided α=0.05 for primary safety endpoint will be performed with the per-protocol safety analysis set and a CMH test at 2-sided α=0.05, stratified by MSKCC and prior PD-1/PD-L1 treatment from IxRS.

Results

Subject Disposition and Reasons for Discontinuation from Study Treatment (see Table 5).

TABLE 5

| | Lenvatinib 14 mg + | Lenvatinib 18 mg + |
|---|---|---|
| | Everolimus | Everolimus |
| | (N = 172) | (N = 171) |
| | n (%) | n (%) |
| Treatment Ongoing at Data | 38 (22.1) | 50 (29.2) |
| Cutoff Date | | |
| On Both Study Drugs | 33 (19.2) | 45 (26.3) |
| On Lenvatinib only | 4 (2.3) | 5 (2.9) |
| On Everolimus only | 1 (0.6) | 0 |
| Discontinued Treatment | 134 (77.9) | 119 (69.6) |
| Primary Reason(s) for | | |
| Discontinuation from the | | |
| Treatment | | |
| Radiological Disease | 78 (45.3) | 64 (37.4) |
| Progression | | |
| Clinical Disease Progression | 15 (8.7) | 6 (3.5) |
| Adverse event | 27 (15.7) | 30 (17.5) |
| Time to Treatment | | |
| Discontinuation due to AE | | |
| (months) | | |
| Median | 3.15 | 5.70 |
| Min, Max | 0.5, 12.7 | 0.8, 24.6 |
| Subject Choice | 5 (2.9) | 7 (4.1) |
| Lost to follow-up | 0 | 1 (0.6) |
| Withdrawal of Consent | 5 (2.9) | 4 (2.3) |
| Other | 4 (2.3) | 7 (4.1) |

Demographic and Baseline Characteristics (see Table 6)

TABLE 6

| | Lenvatinib 14 mg + | Lenvatinib 18 mg + |
|---|---|---|
| | Everolimus | Everolimus |
| Characteristic | (N = 172) | (N = 171) |
| Median age (range), years | 61 (28.82) | 62 (35.87) |
| Sex, male, n (%) | 133 (77.3) | 129 (75.4) |
| KPS Score at Baseline, ≥90 n | 128 (74.4) | 124 (72.5) |
| (%) | | |

MSKCC Prognostic Group from CRF Data, n (%)

| | | |
|---|---|---|
| Favorable Risk | 49 (28.5) | 50 (29.2) |
| Intermediate Risk | 93 (54.1) | 90 (52.6) |
| Poor Risk | 30 (17.4) | 31 (18.1) |
| Prior PD-1/PD-L1 Treatment | 49 (28.5) | 41 (24.0) |
| from CRF Data, Yes, n (%) | | |
| Baseline Hypertension Status, | 118 (68.6) | 123 (71.9) |
| Yes, n (%) | | |

No. of Prior Anti Cancer Therapy Regimens

| | | |
|---|---|---|
| 1 | 129 (75.0) | 140 (81.9) |
| 2 | 38 (22.1) | 29 (17.0) |
| ≥3 | 5 (2.9) | 2 (1.2) |

Important Protocol Deviations (see table 7)

TABLE 7

| | Lenvatinib 14 mg + | Lenvatinib 18 mg + |
|---|---|---|
| | Everolimus | Everolimus |
| | (N = 172) | (N = 171) |
| Type of Deviation | n (%) | n (%) |
| Subjects with at Least One | 27 (15.7) | 19 (11.1) |
| Important Protocol Deviation | | |
| Eligibility Criteria | 4 (2.3) | 1 (0.6) |
| IP Deviation^{a} | 7 (4.1) | 0 |
| Treatment Error^{b} | 16 (9.3) 17 ( | 17 (9.9) |
| Withdrawal Criteria | 0 | 2 (1.2) |

(a): All of these relate to subjects that were not up-titrated to 18 mg at C2 despite no intolerable G2 or G3 AEs. (b) 32 of the 33 are the incorrect Lenvatinib doses due to IxRS issues.

Summary of Tumor Response at 24 weeks—Investigator assessment (Primary Endpoint) (See Table 8).

TABLE 8

| | Lenvatinib 14 mg + | Lenvatinib 18 mg + |
|---|---|---|
| | Everolimus | Everolimus |
| Response Category | (N = 156) | (N = 155) |
| Best Overall Response | | |
| (BOR) as of Week 24, | | |
| n (%) | | |
| Complete Response (CR) | 0 | 1 (0.6) |
| Partial Response (PR) | 50 (32.1) | 53 (34.2) |
| Stable Disease (SD | 85 (54.5) | 81 (52.3) |
| Progressive Disease (PD) | 7 (4.5) | 7 (4.5) |
| Not Evaluable (NE) | 14 (9.0) | 13 (8.4) |
| Objective Response Rate | 50 (32.1) | 54 (34.8) |
| as of Week 24 (CR + PR), | | |
| n (%) | | |
| 95% CI of Objective | (24.7, 39.4) | (27.3, 42.3) |
| Response Rate | | |
| Difference (%) (90% CI) | −2.8 (−11.6, 6.0) | |
| Odds Ratio (90% CI) with | 0.88 (0.59, 1.32) | |
| Stratification Factors | | |
| from IxRS | | |
| P value (1-sided) | 0.2676 | |
| Odds Ratio (90% CI) with | 0.88 (0.59, 1.31) | |
| Stratification Factors | | |
| from CRF | | |
| P value (1-sided) | 0.2787 | |

If 1-sided P value is ≤0.045, non-inferiority in ORR_{24W} by investigator assessment can be claimed.

ORR_{24W} Analysis sets—Investigator Assessment (see Table 9).

TABLE 9

| | Lenvatinib 14 mg + | Lenvatinib 18 mg + |
|---|---|---|
| Response Category | Everolimus | Everolimus |
| PPAS 1 Inv Assessment | 156 | 155 |
| (Primary Population), n | | |
| Objective Response Rate as | 50 (32.1) | 54 (34.8) |
| of Week 24 (CR + PR), | | |
| n (%) | | |
| 95% CI of Objective | (24.7, 39.4) | (27.3, 42.3) |
| Response | | |
| Difference (%) (90% CI) | −2.8 (−11.6, 6.0) | |
| Odds Ratio (90% CI) with | 0.88 (0.59, 1.32) | |
| Stratification Factors | | |
| from IxRS | | |
| FAS Inv Assessment, n | 172 | 171 |
| Objective Response Rate as | 58 (33.7) | 61 (35.7) |
| of Week 24 (CR + PR), | | |
| n (%) | | |
| 95% CI of Objective | (26.7, 40.8) | (28.5, 42.9) |
| Response Rate | | |
| Difference (%) (90% CI) | −2.0 (−4.7, 13.3) | |
| Odds Ratio (90% Cl) with | 0.92 (0.63, 1.33) | |
| Stratification Factors | | |
| from IxRS | | |
| PPAS 2 Inv Assessment, n | 143 | 145 |
| Objective Response Rate as | 47 (32.9) | 53 (36.6) |
| of Week 24 (CR + PR), | | |
| n (%) | | |
| 95% CI of Objective | (25.2, 40.6) | (28.7, 44.4) |
| Response Rate | | |
| Difference (%) (90% CI) | −3.7 (−12.9, 5.5) | |
| Odds Ratio (90% CI) with | 0.83 (0.55, 1.26) | |
| Stratification Factors | | |
| from IxRS | | |

Summary of Tumor Response at 24 weeks—IIR (Exploratory Endpoint) (see Table 10)

TABLE 10

| | Lenvatinib 14 mg + | Lenvatinib 18 mg + |
|---|---|---|
| | Everolimus | Everolimus |
| Response Category | (N = 156) | (N = 155) |
| Best Overall Response | | |
| (BOR) as of Week 24, n | | |
| (%) | | |
| Complete Response (CR) | 3 (1.9) | 4 (2.6) |
| Partial Response (PR) | 58 (37.2) | 50 (32.3) |
| Stable Disease (SD) | 67 (42.9) | 75 (48.4) |
| Progressive Disease (PD) | 8 (5.1) | 8 (5.2) |
| Not Evaluable (NE) | 20 (12.8) | 18 (11.6) |
| Objective Response Rate as | 61 (39.1) | 54 (34.8) |
| of Week 24 (CR + PR), n | | |
| (%) | | |
| 95% CI of Objective | (31.4, 46.8) | (27.3, 42.3) |
| Response Rate | | |
| Difference (%) (90% CI) | 4.3 (−4.7, 13.3) | |
| Odds Ratio (90% CI) with | 1.20 (0.82, 1.76) | |
| Stratification Factors | | |
| from IxRS | | |
| P value (1-sided) | 0.0254 | |
| Odds Ratio (90% CI) with | 1.21 (0.82, 1.78) | |
| Stratification Factors | | |
| from CRF | | |
| P value (1-sided) | 0.0246 | |

If 1-sided P value is ≤0.045, non-inferiority in ORR_{24W} by investigator assessment can be claimed.

ORR_{24W} Analysis sets—IIR Assessment (see Table 11).

TABLE 11

| | Lenvatinib 14 mg + | Lenvatinib 18 mg + |
|---|---|---|
| Response Category | Everolimus | Everolimus |
| PPAS 1 Inv Assessment | 156 | 155 |
| (Primary Population), n | | |
| Objective Response Rate as | 61 (39.1) | 54 (34.8) |
| of Week 24 (CR + PR), | | |
| n (%) | | |
| 95% CI of Objective | (31.4, 46.8) | (27.3, 42.3) |
| Response Rate | | |
| Difference (%) (90% CI) | 4.3 (−4.7, 13.3) | |
| Odds Ratio (90% CI) with | 1.20 (0.82, 1.76) | |
| Stratification Factors | | |
| from IxRS | | |
| FAS Inv Assessment, n | 172 | 171 |
| Objective Response Rate as | 67 (39.0) | 62 (36.3) |
| of Week 24 (CR + PR), | | |
| n (%) | | |
| 95% CI of Objective | (31.7, 46.2) | (29.1, 43.5) |
| Response Rate | | |
| Difference (%) (90% CI) | 2.7 (−5.9, 11.3) | |
| Odds Ratio (90% CI) with | 1.12 (0.78, 1.62) | |
| Stratification Factors | | |
| from IxRS | | |
| PPAS 2 Inv Assessment, n | 143 | 145 |
| Objective Response Rate as | 56 (39.2) | 53 (36.6) |
| of Week 24 (CR + PR), | | |
| n (%) | | |
| 95% CI of Objective | (31.2, 47.2) | (28.7, 44.4) |
| Response Rate | | |
| Difference (%) (90% CI) | 2.6 (−6.8, 12.0) | |
| Odds Ratio (90% CI) with | 1.12 (0.75, 1.66) | |
| Stratification Factors | | |
| from IxRS | | |

Progression Free Survival Based on Investigator Assessment (see Table 12)

TABLE 12

| | Lenvatinib 14 mg + | Lenvatinib 18 mg + |
|---|---|---|
| | Everolimus | Everolimus |
| | (N = 156) | (N = 155) |
| Subjects with Events, n (%) | 85 (54.5) | 63 (40.6) |
| Disease Progression | 67 (42.9) | 54 (34.8) |
| Death | 18 (11.5) | 9 (5.8) |
| Censored Subjects, n (%) | 71 (45.5) | 92 (59.4) |
| No Adequate Post Baseline | 1 (0.6) | 8 (5.2) |
| Tumor Assessment | | |
| Death or Progression after | 4 (2.6) | 0 (0.0) |
| more than One Missing | | |
| Assessments | | |
| New Anti-cancer Treatment | 6 (3.8) | 3 (1.9) |
| Started | | |
| Treatment discontinuation | 14 (9.0) | 20 (12.9) |
| for reasons other than | | |
| progression | | |
| No Progression at the Time | 46 (29.5) | 61 (39.4) |
| of Data Cut-off | | |
| Progression-Free Survival | | |
| (months) | | |
| Median (95% CI) | 11.1 (9.0, 12.9) | 14.7 (11.1, 20.3) |
| Q1 (95% CI) | 5.6 (4.8, 7.2) | 7.3 (5.5, 8.0) |
| Q3 (95% CI) | 18.4 (15.2, NE) | NE (24.0, NE) |
| Stratified Hazard Ratio | 1.42 (1.08, 1.86) | |
| (90% CI) with Stratification | | |
| Factors from IxRS | | |
| Straitified Hazard Ratio | 1.48 (1.11, 1.95) | |
| (90% CI) with Stratification | | |
| Factors from CRF | | |
| Progression-Free Survival | | |
| Rate (%) (95% CI) at | | |
| 6 Months | 69.3 (60.8, 76.4) | 78.8 (70.7, 84.9) |
| 12 Months | 44.2 (34.8, 53.1) | 56.3 (46.4, 65.0) |
| 18Months | 29.2 (20.3, 38.6) | 44.9 (34.4, 54.8) |
| 24 Months | 17.8 (8.6, 29.6) | 29.7 (15.0, 45.9) |

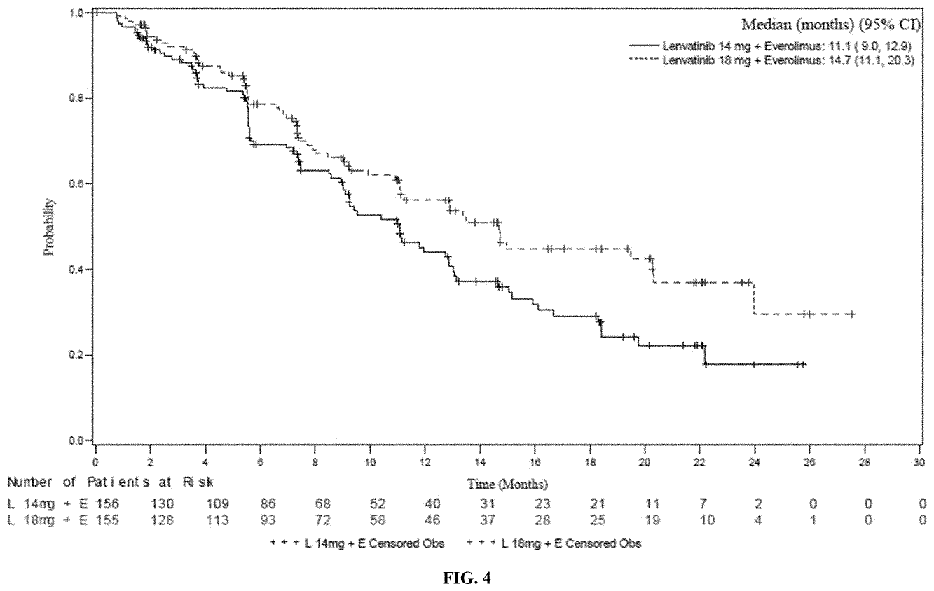

The Progression Free Survival Based on Investigator Assessment can also be viewed in a Kaplan-Meier plot (see FIG. 4).

Progression Free Survival Based on IIR Assessment (see Table 13).

TABLE 13

| | Lenvatinib 14 mg + | Lenvatinib 18 mg + |
|---|---|---|
| | Everolimus | Everolimus |
| | (N = 156) | (N = 155) |
| Subjects with Events, n (%) | 80 (51.3) | 67 (43.2) |
| Disease Progression | 66 (42.3) | 58 (37.4) |
| Death | 14 (9.0) | 9 (5.8) |
| Censored Subjects, n (%) | 76 (48.7) | 88 (56.8) |
| No Adequate Post Baseline | 3 (1.9) | 8 (5.2) |
| Tumor Assessment | | |
| New Anti-cancer Treatment | 10 (6.4) | 6 (3.9) |
| Started | | |
| Treatment discontinuation | 12 (7.7) | 19 (12.3) |
| for reasons other than | | |
| progression | | |
| No Progression at the Time | 51 (32.7) | 55 (35.5) |
| of Data Cut-off | | |
| Progression-Free Survival | | |
| (months) | | |
| Median (95% CI) | 11.0 (9.3, 12.9) | 12.9 (9.2, 17.1) |
| Q1 (95% CI) | 5.6 (4.7, 7.7) | 5.6 (5.4, 7.4) |
| Q3 (95% CI) | 18.4 (14.6, NE) | NE (18.4, NE) |
| Stratified Hazard Ratio | 1.21 (0.92, 1.59) | |
| (90% CI) with Stratification | | |
| Factors from IxRS | | |
| Stratified Hazard Ratio | 1.21 (0.92, 1.59) | |
| (90% CI) with Stratification | | |
| Factors from CRF | | |
| Progression-Free Survival | | |
| Rate (%) (95% CI) at | | |
| 6 Months | 74.1 (65.7, 80.7) | 72.4 (63.7, 79.4) |
| 12 Months | 41.7 (31.9, 51.3) | 50.8 (40.6, 60.0) |
| 18 Months | 25.2 (16.4, 35.0) | 39.3 (28.6, 49.8) |
| 24 Months | 19.5 (11.2, 29.4) | 27.8 (17.0, 39.6) |

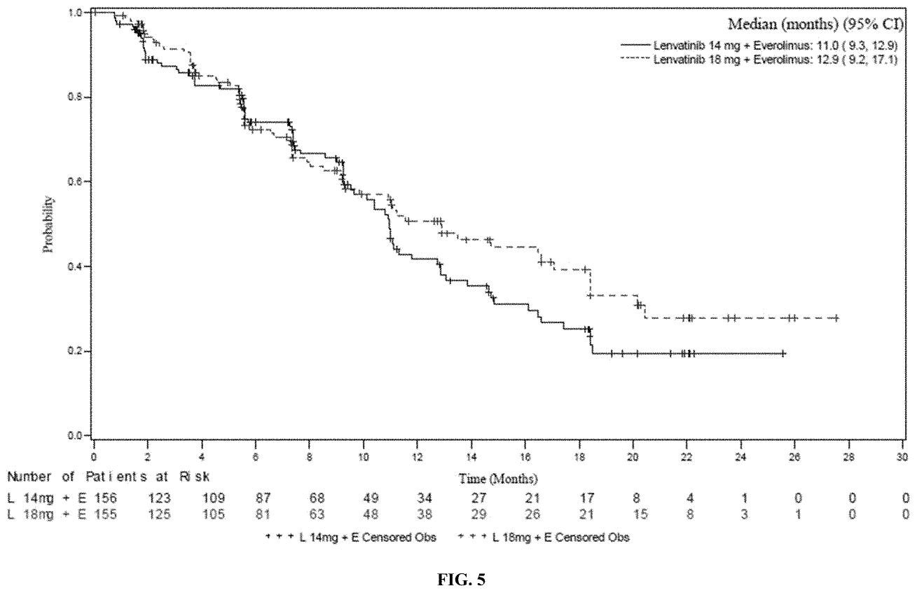

The Progression Free Survival Based on IIR Assessment can also be viewed in a Kaplan-Meier plot (see FIG. 5).

Objective Response Rate, Investigator Assessment (see Table 14).

TABLE 14

| | Lenvatinib 14 mg + | Lenvatinib 18 mg + |
|---|---|---|
| | Everolimus | Everolimus |
| Response Category | (N = 156) | (N = 155) |
| Best Overall Response | | |
| (BOR), n (%) | | |
| Complete Response (CR) | 1 (0.6) | 2 (1.3) |
| Partial Response (PR) | 53 (34.0) | 61 (39.4) |
| Stable Disease (SD) | 81 (51.9) | 72 (46.5) |
| Progressive Disease (PD) | 7 (4.5) | 7 (4.5) |
| Not Evaluable (NE) | 14 (9.0) | 13 (8.4) |
| Objective Response Rate | 54 (34.6) | 63 (40.6) |
| (CR + PR), n (%) | | |
| 95% CI of Objective | (27.1, 42.1) | (32.9, 48.4) |
| Response Rate | | |
| Difference (%) (90% CI) | −6.0 (−15.0, 3.0) | |
| Odds Ratio (90% CI)c with | 0.77 (0.52, 1.14) | |
| Stratification Factors | | |
| from IxRS | | |
| Odds Ratio (90% CI) with | 0.76 (0.52, 1.13) | |
| Stratification Factors | | |
| from CRF | | |
| Duration of Objective | | |
| Response (months) | | |
| Median (95% CI) | 11.5 (7.5, 19.2) | 11.7 (9.1, NE) |

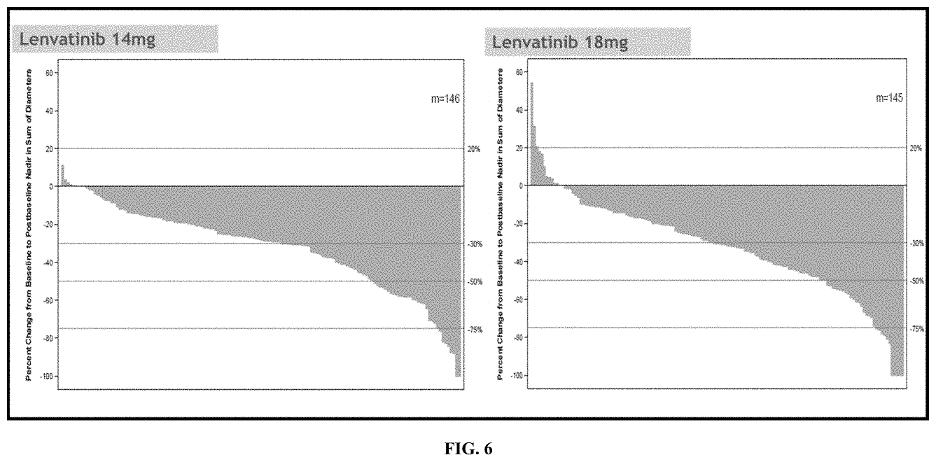

The Percentage Changes in the Sum of Diameters of Target Lesions from both treatment groups has also undergone investigator assessment (see FIG. 6).

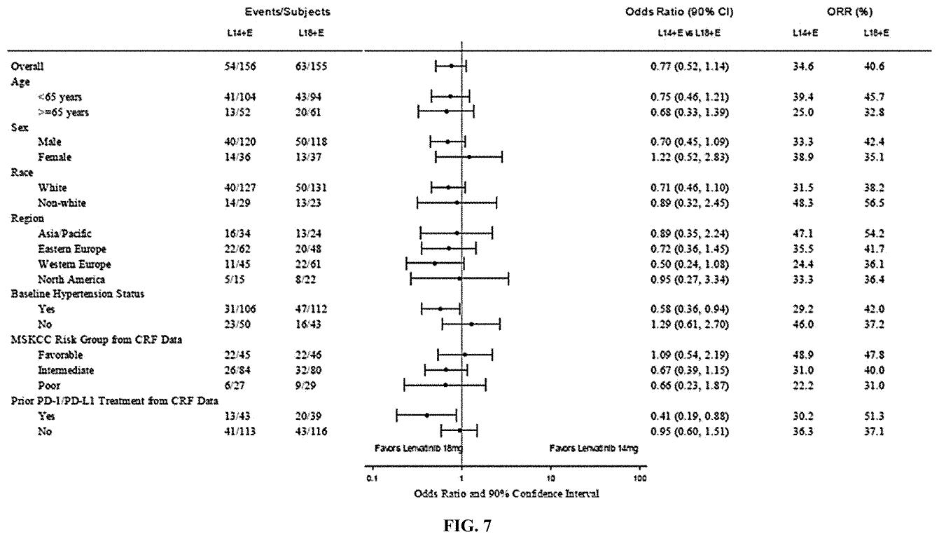

The Subgroup Analysis of the Objective Response Rates for both treatment groups has undergone investigator assessment and the results can be viewed in a forest plot (see FIG. 7).

Objective Response Rate, IIR (see Table 15).

TABLE 15

| | Lenvatinib 14 mg + | Lenvatinib 18 mg + |
|---|---|---|
| | Everolimus | Everolimus |
| Response Category | (N = 156) | (N = 155) |
| Best Overall Response | | |
| (BOR), n (%) | | |
| Complete Response (CR) | 9 (5.8) | 6 (3.9) |
| Partial Response (PR) | 53 (34.0) | 54 (34.8) |
| Stable Disease (SD) | 66 (42.3) | 69 (44.5) |
| Progressive Disease (PD) | 8 (5.1) | 8 (5.2) |
| Not Evaluable (NE) | 20 (12.8) | 18 (11.6) |
| Objective Response Rate | 62 (39.7) | 60 (38.7) |
| (CR + PR), n (%) | | |
| 95% CI of Objective | (32.1, 47.4) | (31.0, 46.4) |
| Response Rate | | |
| Difference (%) (90% CI) | 1.0 (−8.1, 10.1) | |
| Odds Ratio (90% CI)c with | 1.04 (0.71, 1.53) | |
| Stratification Factors | | |
| from IxRS | | |
| Odds Ratio (90% CI) with | 1.05 (0.72, 1.54) | |
| Stratification Factors | | |
| from CRF | | |
| Duration of Objective | | |
| Response (months) | | |
| Median (95% CI) | 11.0 (7.5, 12.9) | 11.7 (7.5, 14.7) |

Summary of Overall Survival (OS) (see Table 16).

TABLE 16

| | Lenvatinib 14 mg + | Lenvatinib 18 mg + |
|---|---|---|
| | Everolimus | Everolimus |
| | (N = 156) | (N = 155) |
| Deaths, n (%) | 59 (37.8) | 51 (32.9) |
| Censored Subjects, n (%) | 97 (62.2) | 104 (67.1) |
| Lost to follow-up | 0 (0.0) | 3 (1.9) |
| Withdrawal of consent | 9 (5.8) | 5 (3.2) |
| Alive | 88 (56.4) | 96 (61.9) |
| Overall Survival (months) | | |
| Median (95% CI) | 27.0 (18.3, NE) | NE (23.8, NE) |
| Q1 (95% CI) | 9.4 (7.0, 13.1) | 11.5 (9.1, 14.0) |
| Q3 (95% CI) | NE (27.0, NE) | NE (NE, NE) |
| Overall Survival Rate (95% | | |
| Cl) | | |
| At 6 Months | 84.4 (77.6, 89.2) | 90.5 (84.5, 94.3) |
| At 12 Months | 69.5 (61.0, 76.5) | 74.3 (66.0, 80.9) |
| At 18 Months | 61.2 (51.8, 69.2) | 61.3 (51.8, 69.5) |
| At 24 Months | 50.2 (39.4, 60.1) | 55.7 (43.3, 66.5) |
| Stratified Hazard Ratio | 1.20 (0.87, 1.65) | |
| (90% CI) with Stratification | | |
| Factors from IxRS | | |
| Stratified Hazard Ratio | 1.24 (0.90, 1.70) | |
| (90% CI) with Stratification | | |
| Factors from CRF | | |

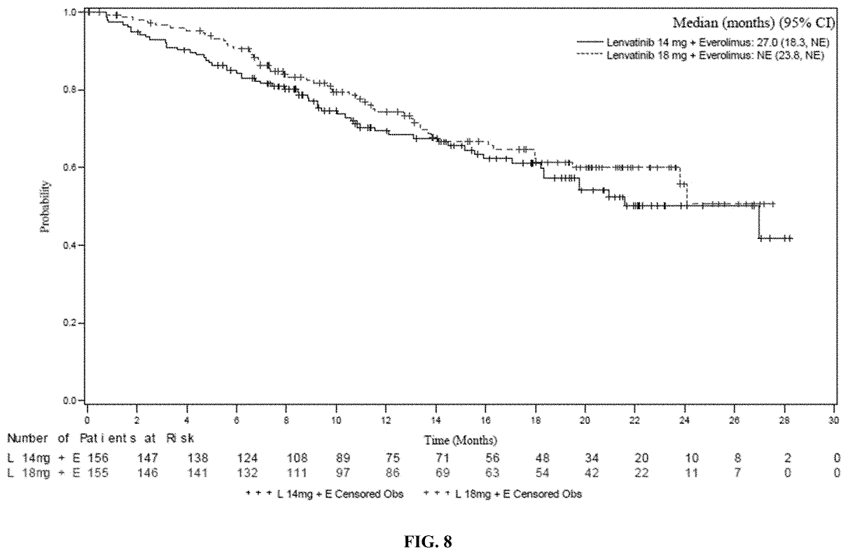

The summarized overall survival results can also be viewed in a Kaplan-Meier plot (see FIG. 8).

Exposure and Dose Intensity (see Table 17).

TABLE 17

| | Lenvatinib 14 mg + | Lenvatinib 18 mg + |
|---|---|---|
| | Everolimus | Everolimus |
| | (N = 173) | (N = 168) |
| Duration of Treatment, | 7.72 | 8.18 |
| median (months) | | |
| Dose Intensity Lenvatinib, | 13.33 | 13.83 |
| median (mg/day) | | |
| Dose Intensity Everolimus, | 4.68 | 4.62 |
| median (mg/day) | | |
| Received Dose as Percentage | 79.99 | 76.82 |
| of Planned Dose, | | |
| Lenvatinib, median | | |
| Received Dose as Percentage | 93.59 | 92.40 |
| of Planned Dose, | | |
| Everolimus, median | | |

Dose modifications of Lenvatinib that occurred within this study (see Table 18).

TABLE 18

| | Lenvatinib 14 mg + | Lenvatinib 18 mg + |
|---|---|---|
| | Everolimus | Everolimus |
| | (N = 173) | (N = 168) |
| Subjects with Dose | 112 (64.7) | 110 (65.5) |
| Reduction, n (%) | | |
| Frequency of Dose | | |
| Reductions, n (%) | | |
| 1 | 54 (31.2) | 49 (29.2) |
| 2 | 39 (22.5) | 39 (23.2) |
| 3 | 19 (11.0) | 20 (1 1.9) |
| ≥4 | 0 | 2 (1.2) |
| Time to First Dose | 2.14 | 2.12 |
| Reduction, median, (months) | | |
| Subjects with Dose | 91 (52.6) | 93 (55.4) |
| Interruption, n (%) | | |
| Frequency of Dose | | |
| Interruptions, n(%) | | |
| 1 | 37 (21.4) | 33 (19.6) |
| 2 | 23 (13.3) | 24 (14.3) |
| 3 | 9 (5.2) | 9 (5.4) |
| ≥4 | 22 (12.7) | 27 (16.1) |
| Time to First Dose | 1.54 | 2.14 |
| Interruption, median, | | |
| (months) | | |

Summary of TEAEs intolerable G2 or ≥G3 after first 24 weeks (Primary Safety Endpoint) (see Table 19).

TABLE 19

| | Lenvatinib 14 mg + | Lenvatinib 18 mg + |
|---|---|---|
| | Everolimus | Everolimus |
| | (N = 157) | (N = 152) |
| Subjects with Intolerable | 130 (82.8) | 121 (79.6) |
| Grade 2 or Any >= Grade 3 | | |
| TEAEs Within 24 Weeks | | |
| After Randomization, n (%) | | |
| 95% CI | 130 (82.8) | (73.2, 86.0) |
| Difference (%) (95% CI) | 3.2 (−5.5, 11.9) | |
| P value Based on | | |
| CMH Method with | 0.4763 | |
| Stratification Factors | | |
| from IxRS | | |
| CMH Method with | 0.4604 | |
| Stratification Factors | | |
| from CRF | | |
| Logistic Regression Model | 0.3556 | |
| with Region and | | |
| Stratification Factors | | |
| from IxRS as Covariates | | |
| Logistic Regression Model | 0.3676 | |
| with Region and | | |
| Stratification Factors | | |
| from CRF as Covariates | | |

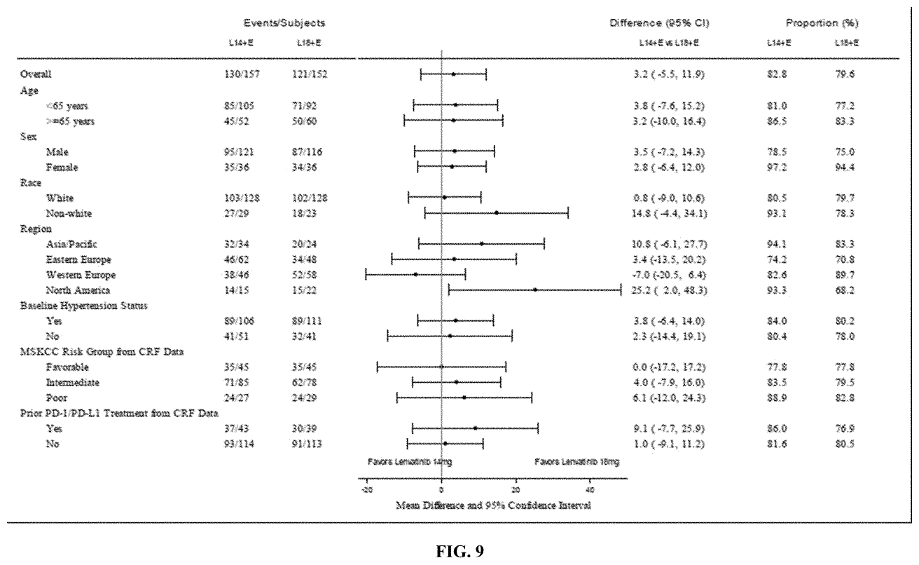

A subgroup analysis has also been performed for subjects with intolerable grade 2 or any ≥grade 3 treatment-emergent adverse events within 24 weeks after randomization (see FIG. 9).

Overview of Treatment-Emergent Adverse Events (see Table 20).

TABLE 20

| | Lenvatinib 14 mg + | Lenvatinib 18 mg + |
|---|---|---|
| | Everolimus | Everolimus |
| Characteristic | (N = 173) | (N = 168) |
| All-Grade AEs, any cause, | 173 (100) | 167 (99.4) |
| n (%) | | |
| Treatment-related all-Grade | 165 (95.4) | 161 (95.8) |
| AEs | | |
| Intolerable Grade 2 AE, n | 15 (8.7) | 11 (6.5) |
| (%) | | |
| Grade 3-4 AE, n (%) | 124 (71.7) | 129 (76.8) |
| Treatment-related Grade | 108 (62.4) | 113 (67.3) |
| 3-4 AE | | |
| Serious adverse event, n | 85 (49.1) | 82 (48.8) |
| (%) | | |
| Grade 5 AE, n (%) | 24 (13.9) | 15 (8.9) |
| Treatment-related Grade 5 | 3 (1.7) | 3 (1.8) |
| AE | | |
| AE leading to study drug | 56 (32.4) | 45 (26.8) |
| discontinuation, n (%) | | |
| AE leading to discontinuation | 45 (26.0) | 37 (22.0) |
| of Lenvatinib, n (%) | | |
| AE leading to discontinuation | 55 (31.8) | 43 (25.6) |
| of Everolimus, n (%) | | |
| AE leading to dose | 129 (74.6) | 140 (83.3) |
| interruption of any study | | |
| treatment, n (%) | | |
| AE leading to interruption | 105 (60.7) | 117 (69.6) |
| of Lenvatinib, n (%) | | |
| AE leading to interruption | 126 (72.8) | 137 (81.5) |
| of Everolimus, n(%) | | |
| AE leading to study drug | 117 (67.6) | 117 (69.6) |
| dose reduction, n (%) | | |
| AE leading to dose reduction | 114 (65.9) | 114 (67. 9) |
| of Lenvatinib, n (%) | | |
| AE leading to dose reduction | 29 (16.8) | 30 (17.9) |
| of Everolimus, n (%) | | |

Treatment-emergent Adverse Events with Frequency>=10% in Either Treatment Arm by System Organ Class and Preferred Term Safety Analysis Set (see Table 21).

TABLE 21

| | Lenvatinib 14 mg + | Lenvatinib 18 mg + | |
|---|---|---|---|
| | Everolimus | Everolimus | Total |
| MedDRA System Organ Class | (N = 173) | (N = 168) | (N = 341) |
| Preferred Term | n (%) | n (%) | n (%) |
| Subjects with Any TEAEs | 173 (100) | 167 (99.4) | 340 (99.7) |
| Blood and lymphatic system disorders | 40 (23.1) | 37 (22.0) | 77 (22.6) |
| Anaemia | 31 (17.9) | 24 (14.3) | 55 (16.1) |
| Endocrine disorders | 34 (19.7) | 39 (23.2) | 73 (21.4) |
| Hypothyroidism | 30 (17.3) | 31 (18.5) | 61 (17.9) |
| Gastrointestinal disorders | 154 (89.0) | 149 (88.7) | 303 (88.9) |
| Diarrhoea | 118 (68.2) | 121 (72.0) | 239 (70.1) |
| Stomatitis | 59 (34.1) | 47 (28.0) | 106 (31.1) |
| Nausea | 53 (30.6) | 52 (31.0) | 105 (30.8) |
| Vomiting | 41 (23.7) | 42 (25.0) | 83 (24.3) |
| Abdominal pain | 28 (16.2) | 26 (15.5) | 54 (15.8) |
| Constipation | 22 (12.7) | 25 (14.9) | 47 (13.8) |
| General disorders and administration site | 113 (65.3) | 107 (63.7) | 220 (64.5) |
| conditions | | | |
| Fatigue | 50 (28.9) | 48 (28.6) | 98 (28.7) |
| Asthenia | 43 (24.9) | 37 (22.0) | 80 (23.5) |
| Oedema peripheral | 19 (11.0) | 25 (14.9) | 44 (12.9) |
| Investigations | 103 (59.5) | 99 (58.9) | 202 (59.2) |
| Weight decreased | 34 (19.7) | 41 (24.4) | 75 (22.0) |
| Blood creatinine increased | 20 (11.6) | 24 (14.3) | 44 (12.9) |
| Lipase increased | 19 (11.0) | 14 (8.3) | 33 (9.7) |
| Blood cholesterol increased | 13 (7.5) | 22 (13.1) | 35 (10.3) |
| Metabolism and nutrition disorders | 116 (67.1) | 117(69.6) | 233 (68.3) |
| Decreased appetite | 61 (35.3) | 58 (34.5) | 119 (34.9) |
| Hypertriglyceridaemia | 35 (20.2) | 37 (22.0) | 72 (21.1) |
| Hypercholesterolaemia | 31 (17.9) | 25 (14.9) | 56 (16.4) |
| Hypokalaemia | 10 (5.8) | 20 (11.9) | 30 (8.8) |
| Musculoskeletal and connective tissue | 69 (39.9) | 65 (38.7) | 134 (39.3) |
| disorders | | | |
| Arthralgia | 21 (12.1) | 16 (9.5) | 37(10.9) |
| Back pain | 16 (9.2) | 17 (10.1) | 33 (9.7) |
| Nervous system disorders | 54 (31.2) | 35 (20.8) | 89 (26.1) |
| Headache | 22 (12.7) | 19 (11.3) | 41 (12.0) |
| Renal and urinary disorders | 62 (35.8) | 78 (46.4) | 140 (41.1) |
| Proteinuria | 39 (22.5) | 60 (35.7) | 99 (29.0) |
| Respiratory, thoracic and mediastinal | 79 (45.7) | 83 (49.4) | 162 (47.5) |
| disorders | | | |
| Epistaxis | 26 (15.0) | 25 (14.9) | 51 (15.0) |
| Dysphonia | 19 (11.0) | 24 (14.3) | 43 (12.6) |
| Dyspnoea | 15 (8.7) | 22 (13.1) | 37 (10.9) |
| Cough | 10 (5.8) | 30 (17.9) | 40 (11.7) |
| Skin and subcutaneous tissue disorders | 67 (38.7) | 76 (45.2) | 143 (41.9) |
| Rash | 27 (15.6) | 28 (16.7) | 55 (16.1) |
| Palmar-plantar erythrodysaesthesia | 23 (13.3) | 26 (15.5) | 49 (14.4) |
| syndrome | | | |
| Vascular disorders | 60 (34.7) | 71 (42.3) | 131 (38.4) |
| Hypertension | 52 (30.1) | 60 (35.7) | 112 (32.8) |

Treatment-emergent Adverse Events of intolerable Grade 2 and Grade>=3 (see Table 22).

TABLE 22

| | Lenvatinib 14 mg + | Lenvatinib 18 mg + | |
|---|---|---|---|
| | Everolimus | Everolimus | Total |
| MedDRA System Organ Class | (N = 157) | (N = 152) | (N = 309) |
| Preferred Term | n (%) | n (%) | n (%) |
| Subjects with Intolerable Grade 2 or Any | 147 (93.6) | 139 (91.4) | 286 (92.6) |
| >= Grade 3 TEAEs | | | |
| Blood and lymphatic system disorders | 16 (10.2) | 11 (7.2) | 27 (8.7) |
| Anaemia | 13 (8.3) | 7 (4.6) | 20 (6.5) |
| Febrile neutropenia | 0 (0.0) | 1 (0.7) | 1 (0.3) |
| Leukocytosis | 0 (0.0) | 1 (0.7) | 1 (0.3) |
| Leukopenia | 1 (0.6) | 0 (0.0) | 1 (0.3) |
| Lymphopenia | 1 (0.6) | 0 (0.0) | 1 (0.3) |
| Splenic vein thrombosis | 1 (0.6) | 0 (0.0) | 1 (0.3) |
| Thrombocytopenia | 3 (1.9) | 2 (1.3) | 5 (1.6) |
| Cardiac disorders | 8 (5.1) | 8 (5.3) | 16 (5.2) |
| Acute myocardial infarction | 0 (0.0) | 2 (1.3) | 2 (0.6) |
| Angina pectoris | 1 (0.6) | 0 (0.0) | 1 (0.3) |
| Atrial fibrillation | 2 (1.3) | 0 (0.0) | 2 (0.6) |
| Atrial flutter | 0 (0.0) | 1 (0.7) | 1 (0.3) |
| Cardiac arrest | 0 (0.0) | 1 (0.7) | 1 (0.3) |
| Cardiac failure | 0 (0.0) | 1 (0.7) | 1 (0.3) |
| Cardiac failure acute | 0 (0.0) | 1 (0.7) | 1 (0.3) |
| Cardiac failure congestive | 1 (0.6) | 0 (0.0) | 1 (0.3) |
| Cardiopulmonary failure | 1 (0.6) | 0 (0.0) | 1 (0.3) |
| Cardiovascular disorder | 1 (0.6) | 0 (0.0) | 1 (0.3) |
| Coronary artery disease | 0 (0.0) | 1 (0.7) | 1 (0.3) |
| Myocardial infarction | 1 (0.6) | 1 (0.7) | 2 (0.6) |
| Ventricular extrasystoles | 1 (0.6) | 0 (0.0) | 1 (0.3) |
| Ear and labyrinth disorders | 0 (0.0) | 2 (1.3) | 2 (0.6) |
| Vertigo | 0 (0.0) | 1 (0.7) | 1 (0.3) |
| Vestibular disorder | 0 (0.0) | 1 (0.7) | 1 (0.3) |
| Endocrine disorders | 1 (0.6) | 3 (2.0) | 4 (1.3) |
| Adrenal insufficiency | 0 (0.0) | 1 (0.7) | 1 (0.3) |
| Glucocorticoid deficiency | 1 (0.6) | 0 (0.0) | 1 (0.3) |
| Hypothyroidism | 0 (0.0) | 1 (0.7) | 1 (0.3) |
| Inappropriate antidiuretic hormone | 0 (0.0) | 1 (0.7) | 1 (0.3) |
| secretion | | | |
| Eye disorders | 1 (0.6) | 2 (1.3) | 3 (1.0) |
| Blindness | 1 (0.6) | 0 (0.0) | 1 (0.3) |
| Vision blurred | 0 (0.0) | 1 (0.7) | 1 (0.3) |
| Vitreous detachment | 0 (0.0) | 1 (0.7) | 1 (0.3) |

Fatal TEAEs>2 observed in subjects participating in the study (see Table 23).

TABLE 23

| | Lenvatinib 14 mg + | Lenvatinib 18 mg + |
|---|---|---|
| | Everolimus | Everolimus |
| | (N = 173) | (N = 168) |
| | n (%) | n (%) |
| Subjects with Any Fatal | 24 (13.9) | 15 (8.9) |
| TEAEs | | |
| Malignant neoplasm | 5 (2.9) | 4 (2.4) |
| progression | | |
| Pneumonia | 3 (1.7) | 1 (0.6) |
| Death | 2 (1.2) | 0 (0.0) |
| Pneumonitis | 2 (1.2) | 0 (0.0) |
| Sepsis | 2 (1.2) | 0 (0.0) |

Treatment-emergent SAEs with Frequency>=2% observed in patients of this study (see Table 24).

TABLE 24

| | Lenvatinib 14 mg + | Lenvatinib 18 mg + |
|---|---|---|
| MedDRA System Organ Class | Everolimus | Everolimus |
| Preferred Term | (N = 173) | (N = 168) |
| Subjects with Any Serious | 85 (49.1) | 82 (48.8) |
| TEAEs | | |
| Gastrointestinal disorders | 22 (12.7) | 14 (8.3) |
| Diarrhea | 11 (6.4) | 5 (3.0) |
| Vomiting | 6 (3.5) | 0 (0.0) |
| General disorders and | 14 (8.1) | 8 (4.8) |
| administration site | | |
| conditions | | |
| Pyrexia | 4 (2.3) | 1 (0.6) |
| Infections and infestations | 29 (16.8) | 25 (14.9) |
| Pneumonia | 11 (6.4) | 7 (4.2) |
| Gastroenteritis | 4 (2.3) | 1 (0.6) |
| Neoplasms benign, malignant | 11 (6.4) | 10 (6.0) |
| and unspecified (incl cysts | | |
| and polyps) | | |
| Malignant neoplasm | 5 (2.9) | 5 (3.0) |
| progression | | |
| Renal and urinary disorders | 5 (2.9) | 12 (7.1) |
| Acute kidney injury | 5 (2.9) | 6 (3.6) |

Overview of Clinically Significant TEAEs for Lenvatinib (see Table 25)

TABLE 25

| | Lenvarinib 14 mg + Everolimus | | | | | Lenvarinib 18 mg + Everolimus | | | | |
|---|---|---|---|---|---|---|---|---|---|---|
| Clinically Significant TEAE | (N = 173) n (%) | | | | | (N = 168) n (%) | | | | |
| MedDRA Preferred Term | Any Grade | Grade 3 | Grade 4 | Grade 5 | Grade >=3 | Any Grade | Grade 3 | Grade 4 | Grade 5 | Grade >=3 |
| Subjects with Any Clinically | 130 (75.1) | 48 (27.7) | 4 (2.3) | 3 (1.7) | 55 (31.8) | 135 (80.4) | 57 (33.9) | 9 (5.4) | 6 (3.6) | 72 (42.9) |
| Significant TEAEs | | | | | | | | | | |
| Arterial TE Events | 3 (1.7) | 1 (0.6) | 0 (0.0) | 0 (0.0) | 1 (0.6) | 5 (3.0) | 3 (1.8) | 1 (0.6) | 1 (0.6) | 5 (3.0) |
| Hemiparesis | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Myocardial infarction | 1 (0.6) | 1 (0.6) | 0 (0.0) | 0 (0.0) | 1 (0.6) | 1 (0.6) | 1 (0.6) | 0 (0.0) | 0 (0.0) | 1 (0.6) |
| Retinal artery occlusion | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Acute myocardial infarction | 0 (0.0) | 0 (0.0) | 0 (0.0) | 0 (0.0) | 0 (0.0) | 2 (1.2) | 1 (0.6) | 1 (0.6) | 0 (0.0) | 2 (1.2) |
| Aortic thrombosis | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Cerebrovascular accident | 0 (0.0) | 0 (0.0) | 0 (0.0) | 0 (0.0) | 0 (0.0) | 1 (0.6) | 0 (0.0) | 0 (0.0) | 1 (0.6) | 1 (0.6) |
| Cardiac Dysfunction | 2 (1.2) | 0 (0.0) | 1 (0.6) | 1 (0.6) | 2 (1.2) | 3 (1.8) | 1 (0.6) | 0 (0.0) | 1 (0.6) | 2 (1.2) |
| Cardiac failure congestive | 1 (0.6) | 0 (0.0) | 1 (0.6) | 0 (0.0) | 1 (0.6) | 0 (0.0) | 0 (0.0) | 0 (0.0) | 0 (0.0) | 0 (0.0) |
| Cardiopulmonary failure | 1 (0.6) | 0 (0.0) | 0 (0.0) | 1 (0.6) | 1 (0.6) | 0 (0.0) | 0 (0.0) | 0 (0.0) | 0 (0.0) | 0 (0.0) |
| Cardiac failure | 0 (0.0) | 0 (0.0) | 0 (0.0) | 0 (0.0) | 0 (0.0) | 1 (0.6) | 0 (0.0) | 0 (0.0) | 1 (0.6) | 1 (0.6) |
| Cardiac failure acute | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Cardiac failure chronic | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| Fistula Formation | 2 (1.2) | 0 (0.0) | 0 (0.0) | 0 (0.0) | 0 (0.0) | 3 (1.8) | 2 (1.2) | 0 (0.0) | 0 (0.0) | 2 (1.2) |
| Anal fistula | 2 (1.2) | 0 (0.0) | 0 (0.0) | 0 (0.0) | 0 (0.0) | 3 (1.8) | 2 (1.2) | 0 (0.0) | 0 (0.0) | 2 (1.2) |
| GI Perforation | 4 (2.3) | 1 (0.6) | 1 (0.6) | 1 (0.6) | 3 (1.7) | 9 (5.4) | 6 (3.6) | 1 (0.6) | 1 (0.6) | 8 (4.8) |
| Anal abscess | 1 (0.6) | 1 (0.6) | 0 (0.0) | 0 (0.0) | 1 (0.6) | 0 (0.0) | 0 (0.0) | 0 (0.0) | 0 (0.0) | 0 (0.0) |
| Appendicitis perforated | 1 (0.6) | 0 (0.0) | 0 (0.0) | 1 (0.6) | 1 (0.6) | 1 (0.6) | 1 (0.6) | 0 (0.0) | 0 (0.0) | 1 (0.6) |
| Perirectal abscess | 1 (0.6) | 0 (0.0) | 0 (0.0) | 0 (0.0) | 0 (0.0) | 1 (0.6) | 1 (0.6) | 0 (0.0) | 0 (0.0) | 1 (0.6) |
| Peritonitis | 1 (0.6) | 0 (0.0) | 1 (0.6) | 0 (0.0) | 1 (0.6) | 2 (1.2) | 1 (0.6) | 1 (0.6) | 0 (0.0) | 2 (1.2) |
| Appendiceal abscess | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Gastric ulcer perforation | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Intestinal perforation | 0 (0.0) | 0 (0.0) | 0 (0.0) | 0 (0.0) | 0 (0.0) | 1 (0.6) | 0 (0.0) | 0 (0.0) | 1 (0.6) | 1 (0.6) |
| Large intestine perforation | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Perineal abscess | 0 (0.0) | 0 (0.0) | 0 (0.0) | 0 (0.0) | 0 (0.0) | 2 (1.2) | 1 (0.6) | 0 (0.0) | 0 (0.0) | 1 (0.6) |
| Small intestinal perforation | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Hemorrhage | 40 (23.1) | 2 (1.2) | 0 (0.0) | 1 (0.6) | 3 (1.7) | 34 (20.2) | 4 (2.4) | 0 (0.0) | 1 (0.6) | 5 (3.0) |
| Epistaxis | 26 (15.0) | 1 (0.6) | 0 (0.0) | 0 (0.0) | 1 (0.6) | 25 (14.9) | 1 (0.6) | 0 (0.0) | 0 (0.0) | 1 (0.6) |
| Haemoptysis | 7 (4.0) | 1 (0.6) | 0 (0.0) | 0 (0.0) | 1 (0.6) | 5 (3.0) | 0 (0.0) | 0 (0.0) | 0 (0.0) | 0 (0.0) |
| Haematuria | 4 (2.3) | 1 (0.6) | 0 (0.0) | 0 (0.0) | 1 (0.6) | 4 (2.4) | 1 (0.6) | 0 (0.0) | 0 (0.0) | 1 (0.6) |
| Gingival bleeding | 3 (1.7) | 0 (0.0) | 0 (0.0) | 0 (0.0) | 0 (0.0) | 1 (0.6) | 0 (0.0) | 0 (0.0) | 0 (0.0) | 0 (0.0) |
| Haematochezia | 2 (1.2) | 0 (0.0) | 0 (0.0) | 0 (0.0) | 0 (0.0) | 1 (0.6) | 0 (0.0) | 0 (0.0) | 0 (0.0) | 0 (0.0) |
| Haemorrhoidal haemorrhage | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Haemothorax | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Mouth haemorrhage | 1 (0.6) | 0 (0.0) | 0 (0.0) | 0 (0.0) | 0 (0.0) | 1 (0.6) | 0 (0.0) | 0 (0.0) | 0 (0.0) | 0 (0.0) |
| Pulmonary haemorrhage | 1 (0.6) | 0 (0.0) | 0 (0.0) | 1 (0.6) | 1 (0.6) | 0 (0.0) | 0 (0.0) | 0 (0.0) | 0 (0.0) | 0 (0.0) |
| Purpura | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Rectal haemorrhage | 1 (0.6) | 0 (0.0) | 0 (0.0) | 0 (0.0) | 0 (0.0) | 1 (0.6) | 0 (0.0) | 0 (0.0) | 0 (0.0) | 0 (0.0) |
| Skin haemorrage | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Upper gastrointestinal haemorrhage | 1 (0.6) | 0 (0.0) | 0 (0.0) | 0 (0.0) | 0 (0.0) | 2 (1.2) | 0 (0.0) | 0 (0.0) | 1 (0.6) | 1 (0.6) |
| Contusion | 0 (0.0) | 0 (0.0) | 0 (0.0) | 0 (0.0) | 0 (0.0) | 2 (1.2) | 0 (0.0) | 0 (0.0) | 0 (0.0) | 0 (0.0) |
| Intraventricular haemorrhage | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Subdural haemorrhage | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Hepatotoxicity | 25 (14.5) | 6 (3.5) | 0 (0.0) | 0 (0.0) | 6 (3.5) | 24 (14.3) | 10 (6.0) | 0 (0.0) | 1 (0.6) | 11 (6.5) |
| Aspartate aminotransferase | 12 (6.9) | 3 (1.7) | 0 (0.0) | 0 (0.0) | 3 (1.7) | 11 (6.5) | 2 (1.2) | 0 (0.0) | 0 (0.0) | 2 (1.2) |
| increased | | | | | | | | | | |
| Alanine aminotransferase | 10 (5.8) | 2 (1.2) | 0 (0.0) | 0 (0.0) | 2 (1.2) | 13 (7.7) | 5 (3.0) | 0 (0.0) | 0 (0.0) | 5 (3.0) |
| increased | | | | | | | | | | |
| Gamma-glutamyltransferase | 4 (2.3) | 2 (1.2) | 0 (0.0) | 0 (0.0) | 2 (1.2) | 2 (1.2) | 2 (1.2) | 0 (0.0) | 0 (0.0) | 2 (1.2) |
| increased | | | | | | | | | | |
| Ascites | 2 (1.2) | 0 (0.0) | 0 (0.0) | 0 (0.0) | 0 (0.0) | 1 (0.6) | 0 (0.0) | 0 (0.0) | 0 (0.0) | 0 (0.0) |
| Hepatic function abnormal | 2 (1.2) | 0 (0.0) | 0 (0.0) | 0 (0.0) | 0 (0.0) | 0 (0.0) | 0 (0.0) | 0 (0.0) | 0 (0.0) | 0 (0.0) |
| Transaminases increased | 2 (1.2) | 0 (0.0) | 0 (0.0) | 0 (0.0) | 0 (0.0) | 3 (1.8) | 0 (0.0) | 0 (0.0) | 0 (0.0) | 0 (0.0) |
| Hepatotoxicity (cont.) | | | | | | | | | | |
| Bilirubin conjugated increased | 1 (0.6) | 0 (0.0) | 0 (0.0) | 0 (0.0) | 0 (0.0) | 1 (0.6) | 0 (0.0) | 0 (0.0) | 0 (0.0) | 0 (0.0) |
| Blood bilirubin increased | 1 (0.6) | 0 (0.0) | 0 (0.0) | 0 (0.0) | 0 (0.0) | 3 (1.8) | 1 (0.6) | 0 (0.0) | 0 (0.0) | 1 (0.6) |
| Hepatotoxicity | 1 (0.6) | 1 (0.6) | 0 (0.0) | 0 (0.0) | 1 (0.6) | 0 (0.0) | 0 (0.0) | 0 (0.0) | 0 (0.0) | 0 (0.0) |
| Transaminases abnormal | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Hepatic failure | 0 (0.0) | 0 (0.0) | 0 (0.0) | 0 (0.0) | 0 (0.0) | 1 (0.6) | 0 (0.0) | 0 (0.0) | 1 (0.6) | 1 (0.6) |
| Hepatitis | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Hepatocellular injury | 0 (0.0) | 0 (0.0) | 0 (0.0) | 0 (0.0) | 0 (0.0) | 2 (1.2) | 2 (1.2) | 0 (0.0) | 0 (0.0) | 2 (1.2) |
| Hyperbilirubinaemia | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| Hypertransaminasaemia | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| International normalised ratio | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| increased | | | | | | | | | | |
| Hypertension | 54 (31.2) | 20 (11.6) | 0 (0.0) | 0 (0.0) | 20 (11.6) | 61 (36.3) | 26 (15.5) | 0 (0.0) | 0 (0.0) | 26 (15.5) |
| Hypertension | 52 (30.1) | 18 (10.4) | 0 (0.0) | 0 (0.0) | 18 (10.4) | 60 (35.7) | 25 (14.9) | 0 (0.0) | 0 (0.0) | 25 (14.9) |
| Blood pressure increased | 3 (1.7) | 1 (0.6) | 0 (0.0) | 0 (0.0) | 1 (0.6) | 1 (0.6) | 1 (0.6) | 0 (0.0) | 0 (0.0) | 1 (0.6) |
| Blood pressure systolic | 1 (0.6) | 1 (0.6) | 0 (0.0) | 0 (0.0) | 1 (0.6) | 0 (0.0) | 0 (0.0) | 0 (0.0) | 0 (0.0) | 0 (0.0) |
| increased | | | | | | | | | | |
| Hypocalcemia | 7 (4.0) | 3 (1.7) | 0 (0.0) | 0 (0.0) | 3 (1.7) | 13 (7.7) | 3 (1.8) | 2 (1.2) | 0 (0.0) | 5 (3.0) |
| Hypocalcaemia | 7 (4.0) | 3 (1.7) | 0 (0.0) | 0 (0.0) | 3 (1.7) | 12 (7.1) | 3 (1.8) | 2 (1.2) | 0 (0.0) | 5 (3.0) |
| Blood calcium decreased | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| Hypothyroidism | 38 (22.0) | 0 (0.0) | 0 (0.0) | 0 (0.0) | 0 (0.0) | 35 (20.8) | 1 (0.6) | 0 (0.0) | 0 (0.0) | 1 (0.6) |
| Hypothyroidism | 30 (17.3) | 0 (0.0) | 0 (0.0) | 0 (0.0) | 0 (0.0) | 31 (18.5) | 0 (0.0) | 0 (0.0) | 0 (0.0) | 0 (0.0) |
| Blood thyroid stimulating | 9 (5.2) | 0 (0.0) | 0 (0.0) | 0 (0.0) | 0 (0.0) | 5 (3.0) | 1 (0.6) | 0 (0.0) | 0 (0.0) | 1 (0.6) |
| hormone increased | | | | | | | | | | |
| Autoimmune hypothyroidism | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Palmar-plantar | 23 (13.3) | 3 (1.7) | 0 (0.0) | 0 (0.0) | 3 (1.7) | 27 (16.1) | 0 (0.0) | 0 (0.0) | 0 (0.0) | 0 (0.0) |
| Erythrodysesthesia | | | | | | | | | | |
| Palmar-plantar | 23 (13.3) | 3 (1.7) | 0 (0.0) | 0 (0.0) | 3 (1.7) | 26 (15.5) | 0 (0.0) | 0 (0.0) | 0 (0.0) | 0 (0.0) |
| erythrodysaesthesia | | | | | | | | | | |
| syndrome | | | | | | | | | | |
| Skin reaction | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| Posterior Reversible | 0 (0.0) | 0 (0.0) | 0 (0.0) | 0 (0.0) | 0 (0.0) | 0 (0.0) | 0 (0.0) | 0 (0.0) | 0 (0.0) | 0 (0.0) |
| Encephalopathy Syndrome | | | | | | | | | | |
| Proteinuria | 39 (22.5) | 13 (7.5) | 0 (0.0) | 0 (0.0) | 13 (7.5) | 61 (36.3) | 16 (9.5) | 1 (0.6) | 0 (0.0) | 17 (10.1) |
| Proteinuria | 39 (22.5) | 13 (7.5) | 0 (0.0) | 0 (0.0) | 13 (7.5) | 60 (35.7) | 16 (9.5) | 1 (0.6) | 0 (0.0) | 17 (10.1) |
| Microalbuminuria | 0 (0.0) | 0 (0.0) | 0 (0.0) | 0 (0.0) | 0 (0.0) | 1 (0.6) | 0 (0.0) | 0 (0.0) | 0 (0.0) | 0 (0.0) |
| QT Prolongation | 2 (1.2) | 0 (0.0) | 0 (0.0) | 0 (0.0) | 0 (0.0) | 5 (3.0) | 1 (0.6) | 0 (0.0) | 0 (0.0) | 1 (0.6) |
| Electrocardiogram QT | 2 (1.2) | 0 (0.0) | 0 (0.0) | 0 (0.0) | 0 (0.0) | 5 (3.0) | 1 (0.6) | 0 (0.0) | 0 (0.0) | 1 (0.6) |
| prolonged | | | | | | | | | | |
| Renal Events | 29 (16.8) | 5 (2.9) | 2 (1.2) | 0 (0.0) | 7 (4.0) | 35 (20.8) | 5 (3.0) | 4 (2.4) | 1 (0.6) | 10 (6.0) |
| Blood creatinine increased | 20 (11.6) | 2 (1.2) | 1 (0.6) | 0 (0.0) | 3 (1.7) | 24 (14.3) | 2 (1.2) | 1 (0.6) | 0 (0.0) | 3 (1.8) |
| Acute kidney injury | 7 (4.0) | 3 (1.7) | 0 (0.0) | 0 (0.0) | 3 (1.7) | 10 (6.0) | 3 (1.8) | 2 (1.2) | 1 (0.6) | 6 (3.6) |
| Blood urea increased | 3 (1.7) | 0 (0.0) | 0 (0.0) | 0 (0.0) | 0 (0.0) | 1 (0.6) | 0 (0.0) | 0 (0.0) | 0 (0.0) | 0 (0.0) |
| Renal impairment | 3 (1.7) | 0 (0.0) | 0 (0.0) | 0 (0.0) | 0 (0.0) | 1 (0.6) | 0 (0.0) | 0 (0.0) | 0 (0.0) | 0 (0.0) |
| Renal failure | 2 (1.2) | 0 (0.0) | 1 (0.6) | 0 (0.0) | 1 (0.6) | 4 (2.4) | 1 (0.6) | 0 (0.0) | 0 (0.0) | 1 (0.6) |
| Azotaemia | 1 (0.6) | 0 (0.0) | 0 (0.0) | 0 (0.0) | 0 (0.0) | 2 (1.2) | 0 (0.0) | 0 (0.0) | 0 (0.0) | 0 (0.0) |
| Creatinine renal clearance | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| decreased | | | | | | | | | | |
| Hypercreatininaemia | 1 (0.6) | 0 (0.0) | 0 (0.0) | 0 (0.0) | 0 (0.0) | 0 (0.0) | 0 (0.0) | 0 (0.0) | 0 (0.0) | 0 (0.0) |
| Nephropathy toxic | 0 (0.0) | 0 (0.0) | 0 (0.0) | 0 (0.0) | 0 (0.0) | 1 (0.6) | 1 (0.6) | 0 (0.0) | 0 (0.0) | 1 (0.6) |
| Tubulointerstitial nephritis | 0 (0.0) | 0 (0.0) | 0 (0.0) | 0 (0.0) | 0 (0.0) | 1 (0.6) | 0 (0.0) | 1 (0.6) | 0 (0.0) | 1 (0.6) |

### CONCLUSIONS

Primary Efficacy Endpoint demonstrated that non-inferiority cannot be claimed for the starting dose of lenvatinib 14 mg vs lenvatinib 18 mg both in combination with everolimus. ORR_{24W} (inv assessment)=32.1% vs. 34.8%; OR 0.88; p value 0.2676.

Exploratory endpoint of ORR_{24W} by Independent Review demonstrates a numerical benefit for the starting dose of lenvatinib 14 mg vs lenvatinib 18 mg both in combination with everolimus. ORR_{24W} (inv assessment)=39.1% vs. 34.8%; OR 1.20; p value 0.0254.

Overall this study lead to the discovery that a starting dose of 14 mg of lenvatinib in combination with 5 mg of everolimus leads to an improved dosing regimen for the treatment of RCC.

We claim:

1. A method for treating renal cell carcinoma or advanced renal cell carcinoma, comprising:
administering to a human subject in need of treatment for renal cell carcinoma or advanced renal cell carcinoma a first dosage regimen comprising (i) lenvatinib or a pharmaceutically acceptable salt thereof at a dose of 14 mg/day and (ii) everolimus at a dose of 5 mg/day;
terminating administration of the first dosage regimen after the occurrence of a Grade 3 hypertension until the Grade 3 hypertension is controlled and lowered to at least Grade 2 hypertension; and
administering to the human subject a second dosage regimen comprising lenvatinib or a pharmaceutically acceptable salt thereof at a dose of 10 mg/day once the Grade 3 hypertension has been controlled or lowered.

2. The method of claim 1, wherein the method further comprises terminating administration of the second dosage regimen after occurrence of a second Grade 3 hypertension; and
administering to the human subject a third dosage regimen comprising lenvatinib or a pharmaceutically acceptable salt thereof at a dose of 8 mg/day once the second Grade 3 hypertension has been controlled or lowered to at least Grade 2 hypertension.

3. The method of claim 2, further comprising terminating administration of the third dosage regimen after the occurrence of a third Grade 3 hypertension; and
administering to the human subject a fourth dosage regimen comprising lenvatinib or a pharmaceutically acceptable salt thereof at a dose of 4 mg/day once the third Grade 3 hypertension has been controlled or lowered.

4. The method of claim 3, further comprising terminating administration of the fourth dosage regimen after the occurrence of a fourth Grade 3 hypertension.

5. The method of claim 1, wherein the second dosage regimen comprises everolimus at a dose of 5 mg/day.

6. The method of claim 1, wherein the second dosage regimen comprises everolimus at a dose of 5 mg every other day.

7. The method of claim 1, wherein lenvatinib or the pharmaceutically acceptable salt thereof and everolimus are administered to the human subject orally.

8. The method of claim 1, wherein the human subject has received a prior vascular endothelial growth factor (VEGF)-targeted therapy.

9. A method for treating renal cell carcinoma or advanced renal cell carcinoma, comprising:
administering to a human subject in need of treatment for renal cell carcinoma or advanced renal cell carcinoma a first dosage regimen comprising (i) lenvatinib or a pharmaceutically acceptable salt thereof at a dose of 14 mg/day and (ii) everolimus at a dose of 5 mg/day;
terminating administration of the first dosage regimen after the occurrence of a 2 g or greater proteinuria per 24 hours in the human subject; and
administering to the human subject a second dosage regimen comprising lenvatinib or a pharmaceutically acceptable salt thereof at a dose of 10 mg/day once the proteinuria is less than 2 g per 24 hours.

10. The method of claim 9, further comprising terminating administration of the second dosage regimen after the occurrence of a second 2 g or greater proteinuria per 24 hours; and
administering to the human subject a third dosage regimen comprising lenvatinib or a pharmaceutically acceptable salt thereof at a dose of 8 mg/day once the proteinuria is less than 2 g per 24 hours.

11. The method of claim 10, further comprising terminating administration of the third dosage regimen after the occurrence of a third 2 g or greater proteinuria per 24 hours; and
administering to the human subject a fourth dosage regimen comprising lenvatinib or a pharmaceutically acceptable salt thereof at a dose of 4 mg/day once the proteinuria is less than 2 g per 24 hours.

12. The method of claim 11, further comprising terminating administration of the fourth dosage regimen after the occurrence of a fourth 2 g or greater proteinuria per 24 hours.

13. The method of claim 9, wherein the second dosage regimen comprises everolimus at a dose of 5 mg/day.

14. The method of claim 9, wherein the second dosage regimen comprises everolimus at a dose of 5 mg every other day.

15. The method of claim 9, wherein lenvatinib or the pharmaceutically acceptable salt thereof and everolimus are administered to the human subject orally.

16. The method of claim 9, wherein the human subject has received a prior VEGF-targeted therapy.
